(19) United States
(12) Patent Application Publication
(10) Pub. No.: US 2025/0017553 A1
(43) Pub. Date: Jan. 16, 2025

(54) NEUROVASCULAR ULTRASOUND ANALYSIS FOR ALLERGY TESTING

(71) Applicant: LOMA LINDA UNIVERSITY, Loma Linda, CA (US)

(72) Inventor: Mark Bussell, Loma Linda, CA (US)

(73) Assignee: LOMA LINDA UNIVERSITY, Loma Linda, CA (US)

(21) Appl. No.: 18/716,216

(22) PCT Filed: Dec. 6, 2022
(86) PCT No.: PCT/US22/81044

§ 371 Date: Jun. 4, 2024

Related U.S. Application Data
(60) Provisional application No. 63/265,028, filed on Dec. 6, 2021.

(51) Int. Cl. A61B 8/06; A61B 8/08

(52) U.S. Cl. CPC A61B 8/06; A61B 8/488; A61B 8/5207; A61B 8/5223

(57) ABSTRACT

Computer-implemented methods of neurovascular ultrasound analysis are disclosed for determination of a presence an allergic reaction in a subject. The methods include transmitting an ultrasound wave to an area when an ultrasound probe is positioned on to the area of a subject following exposure of the subject to a potential allergen, generating information describing a change in a circulatory system of the subject based on the Doppler information received by the probe, and detecting a food allergy based on processing the information. Non-transitory computer-readable media and apparatuses for methods of the present disclosure are also disclosed.

### CROSS-REFERENCE TO RELATED APPLICATIONS

This application claims priority to and the benefit of U.S. Provisional Application No. 63/265,028, filed Dec. 6, 2021. The contents of the referenced application are incorporated into the present application by reference herein in its entirety.

### TECHNICAL FIELD

The present disclosure relates to neurovascular ultrasound analysis and, more particularly, to methods, systems, and apparatuses directed to evaluating an allergy using neurovascular ultrasound analysis.

### BACKGROUND

Allergy is an immune system reaction to a foreign substance that occurs following consumption of or exposure to that foreign substance, also known as an allergen. The allergen usually does not cause a reaction in most people and may be an environmental substance, such as pollen, dust, or pet dander. In some instances, the allergen may be a food substance, like peanuts, sesame, dairy, etc. An allergic reaction to an allergen can be unpleasant, a minor irritation, or a life-threatening emergency, depending on a severity of the immune system reaction. A trace amount of an allergen, especially an ingested food allergen, can trigger unpleasant and sometimes life-threatening signs and symptoms, including digestive problems, hives, swollen airways, anaphylaxis, and/or shock (i.e., systemic lowering of blood pressure). Food allergy affects an estimated 6 to 8 percent of children under age 3 and up to 3 percent of adults. While there is no known cure, some children outgrow their food allergy as they get older. Further, a food allergy can be easily misdiagnosed as a food intolerance, which does not involve the immune system reaction and is a less serious condition compared to a food allergy. As such, accurate diagnosis of food allergy or elimination of allergy as the cause of a subject's symptoms is important for a subject's health and well-being. Clinicians consider a number of factors before making an allergy diagnosis in a subject, including the history of food exposure and symptoms, family history of allergies, physical examination, a skin test, a blood IgE test, elimination diet, and/or oral food challenge. However, currently available testing procedures cannot definitively confirm or disprove a food allergy, especially with relative ease. Accordingly, there is a need for methods and systems for effective and reliable detection of allergy to food or environmental allergens in subjects.

### SUMMARY

Computer-implemented methods of neurovascular imaging and ultrasound (US) analysis for determining a presence of an allergic reaction are disclosed. Non-transitory computer-readable media and apparatuses for methods of the present disclosure are also disclosed.

In some aspects, the present disclosure provides a neurovascular US analysis system for determining a presence of an allergic reaction in a subject. The neurovascular US analysis system includes a US probe configured to transmit a US wave to an area of the subject. The area includes an internal body structure. The neurovascular US analysis system includes a processor communicatively coupled to the US probe. The processor is configured to execute instructions to receive, via the US probe, a first information describing a reflection of the US wave from the area, generate a second information describing the internal body structure or a characteristic thereof based on processing the first information, generate a third information describing a change in a circulatory system of the subject based on processing the second information, determine the presence of the allergic reaction to a potential allergen in the subject based on the third information, and output, via a user interface communicatively coupled to the processor, a fourth information indicative of the presence of the allergic reaction.

In some embodiments, the third information includes one or more of fifth information describing a change in an immune response associated with the circulatory system, sixth information describing an amount of compensation on the circulatory system, and seventh information describing a change in vascular circulation by the circulatory system. In some embodiments, the processor is configured to execute the instructions to generate the second information by determining, for an extremity associated with the area, at least one of an anterograde pulsatility index, an anterograde volume flow, a retrograde pulsatility index, and a retrograde volume flow, and determining a neurovascular index for the extremity composed of the at least one of the anterograde pulsatility index, the anterograde volume flow, the retrograde pulsatility index, and the retrograde volume flow. In some embodiments, the processor is configured to execute the instructions to determine an arteriovenous (AV) shunting score for the extremity based on the neurovascular index and a predetermined extremity score. In some embodiments, the processor is configured to execute the instructions to determine a cumulative anterograde volume flow score based on a sum of respective anterograde volume flows of the extremity and at least one additional extremity of the subject. In some embodiments, the processor is configured to execute the instructions to generate the third information by determining a decrease in the cumulative anterograde volume flow score and generating fifth information describing a change in an immune response associated with the circulatory system based on the decrease in the cumulative anterograde volume flow score.

In some embodiments, the processor is configured to execute the instructions to generate the third information by determining an increase in an amount of compensation on the circulatory system and generating sixth information describing the amount of compensation on the circulatory system based on the increase in the amount of compensation on the circulatory system. In some embodiments, the processor is configured to execute the instructions to generate the third information by determining a decrease in an amount of the internal body structure or the characteristic thereof circulating through the circulatory system and generating seventh information describing a change in vascular circulation by the circulatory system based on the decrease in the amount of the internal body structure or the characteristic thereof circulating through the circulatory system.

In some embodiments, the processor is configured to output the fourth information indicative of the presence of the allergic reaction following exposure of the subject to the potential allergen. In some embodiments, the processor is configured to output a pre-exposure fourth information indicative of the presence of the allergic reaction prior to exposure of the subject to the potential allergen to establish baseline measurements and output the fourth information indicative of the presence of the allergic reaction following exposure of the subject to the potential allergen and based on the baseline measurements and post-exposure measurements.

In some embodiments, the area of the subject includes a first area of a plurality of tested areas of the subject. The plurality of tested areas can include a first area and a second area on a radial side of an arm, a first area and a second area on an ulnar side of the arm, a first area, a second area, and a third area on a lower medial side of a leg, and a first area, a second area, and a third area on a lower lateral side of the leg. In some embodiments, the internal body structure includes an artery associated with the area of the subject. In some embodiments, the area includes an upper extremity of the subject or a lower extremity of the subject. The upper extremity can include one or more of a proximal radius, a proximal ulna, a distal radius, and a distal ulna, and the lower extremity can include one or more of a proximal tibia, a proximal peroneal internal body structure, a distal tibia, and a distal peroneal internal body structure.

In some aspects, the present disclosure provides a non-transitory computer-readable medium containing instructions that are executable by a processor to perform operations for neurovascular ultrasound (US) analysis to determine a presence of an allergic reaction. The operations include instructing, via the processor, a US probe communicatively coupled to the processor to transmit a US wave to an area of a subject. The area includes an internal body structure. The operations include receiving, via the processor, a first information describing a reflection of the US wave from the area, generating, via the processor, a second information describing the internal body structure or a characteristic thereof based on processing the first information, generating, via the processor, a third information describing a change in a circulatory system of the subject based on processing the second information, determining, via the processor, the presence of the allergic reaction to a potential allergen in the subject based on the third information, and instructing, via the processor, a user interface communicatively coupled to the processor to output a fourth information indicative of the presence of the allergic reaction.

In some embodiments, the third information includes one or more of fifth information describing a change in an immune response associated with the circulatory system, sixth information describing an amount of compensation on the circulatory system, and seventh information describing a change in vascular circulation by the circulatory system. In some embodiments, determining the presence of the allergic reaction includes determining the one or more of the fifth information, the sixth information, and the seventh information is greater than one or more respective thresholds. In some embodiments, generating the second information describing the internal body structure or the characteristic thereof includes determining, for one or more extremities of the subject, an anterograde pulsatility index, an anterograde volume flow, a retrograde pulsatility index, a retrograde volume flow, or a combination thereof and determining a neurovascular index for the one or more extremities composed of the anterograde pulsatility index, the anterograde volume flow, the retrograde pulsatility index, the retrograde volume flow, or the combination thereof. In some embodiments, the one or more extremities includes an upper extremity or a lower extremity. In some embodiments, generating the third information includes determining a decrease in a cumulative anterograde volume flow score based on a sum of respective anterograde volume flows of the one or more extremities and generating fifth information describing a change in an immune response associated with the circulatory system based on the decrease in the cumulative anterograde volume flow score. In some embodiments, the third information includes the fifth information.

In some embodiments, generating the third information includes determining an increase in an amount of compensation on the circulatory system and generating sixth information describing the amount of compensation based on the increase in the amount of compensation on the circulatory system. In some embodiments, the third information includes the sixth information. In some embodiments, generating the third information includes determining a decrease in an amount of the internal body structure or the characteristic thereof circulating through the circulatory system and generating seventh information describing a change in vascular circulation based on the decrease in the amount of the internal body structure or the characteristic thereof. In some embodiments, the third information includes the seventh information.

In some aspects, the present disclosure provides a computer-implemented method of neurovascular ultrasound (US) analysis (NUA) for determining a presence of an allergic reaction in a subject. In some embodiments, the computer-implemented method includes instructing, via a processor, a US probe communicatively coupled to the processor to transmit a US wave to an area of the subject. The area includes an internal body structure. The computer-implemented method includes receiving, via the processor, a first information describing a reflection of the US wave from the area, generating, via the processor, a second information describing the internal body structure or a characteristic thereof based on processing the first information, and generating, via the processor, a third information describing a change in a circulatory system of the subject based on processing the second information. The third information includes one or more of information describing a change in an immune response associated with the circulatory system, information describing an amount of compensation on the circulatory system, and information describing a change in vascular circulation. The computer-implemented method includes determining, via the processor, the presence of the allergic reaction to a potential allergen in the subject based on the third information and instructing, via the processor, a user interface communicatively coupled to the processor to output a fourth information indicative of the presence of the allergic reaction.

In some embodiments, determining the presence of the allergic reaction includes determining the one or more of the information describing the change in the immune response, the information describing the amount of compensation, and the information describing the change in vascular circulation is greater than one or more respective thresholds.

Other aspects and features of the present disclosure will become apparent to those of ordinary skill in the art after reading the detailed description herein and the accompanying figures.

### BRIEF DESCRIPTION OF THE DRAWINGS

Embodiments described herein are illustrated by examples and not limitations in the accompanying drawings, in which like references indicate similar features. Furthermore, in the drawings some conventional details have been omitted so as not to obscure the inventive concepts described herein.

FIG. 1 illustrates a schematic example of a computer-implemented system for neurovascular ultrasound analysis for food allergy detection, according to an embodiment of the present disclosure.

FIG. 2 illustrates an example of a computer-implemented method for neurovascular ultrasound analysis for food allergy detection in a subject, according to an embodiment of the present disclosure.

FIG. 3A illustrates an example of a computer-implemented method for generating information related to neurovascular ultrasound analysis for food allergy detection, in particular, a method for generating second information describing an internal body structure or a characteristic thereof based on processing first information describing a reflection of an ultrasound wave, according to an embodiment of the present disclosure.

FIG. 3B illustrates an example of a computer-implemented method for generating information related to neurovascular ultrasound analysis for food allergy detection, in particular, another method for generating second information describing an internal body structure or a characteristic thereof based on processing first information describing a reflection of an ultrasound wave, according to an embodiment of the present disclosure.

FIG. 4A illustrates an example of a computer-implemented method for generating information related to neurovascular ultrasound analysis for food allergy detection, in particular, a method for generating third information describing a change in a circulatory system of the subject based on processing the second information, according to an embodiment of the present disclosure.

FIG. 4B illustrates an example of a computer-implemented method for generating information related to neurovascular ultrasound analysis for food allergy detection, in particular, another method for generating the third information describing another change in the circulatory system of the subject based on processing the second information, according to an embodiment of the present disclosure.

FIG. 4C illustrates an example of a computer-implemented method for generating information related to neurovascular ultrasound analysis for food allergy detection, in particular, another method for generating the third information describing another change in the circulatory system of the subject based on processing the second information, according to an embodiment of the present disclosure.

FIG. 5 illustrates an example of a network architecture used in a system for computer-implemented method for neurovascular ultrasound analysis for food allergy detection, according to an embodiment of the present disclosure.

FIG. 6 illustrates an example of an apparatus providing a computer-implemented method for neurovascular ultrasound analysis for food allergy detection, according to an embodiment of the present disclosure.

FIG. 7 illustrates an example of a network architecture used in a system for computer-supported neurovascular ultrasound analysis for vascular changes, according to an embodiment of the present disclosure.

While the disclosure will be described in connection with certain specific embodiments, it will be understood that it is not intended to limit the disclosure to that embodiment. On the contrary, it is intended to cover all alternatives, modifications, and equivalents, as may be included within the spirit and scope of the disclosure as defined by the appended claims.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

As introduced above, it is particularly desirable to provide methods and systems for effective and reliable detection of allergy in subjects. Indeed, allergens can create cumulative trauma to a neurovascular system, such as partial anaphylaxis. This trauma may lead to mastocytosis, causing immune hyper-responsiveness to future perceived threats. Diffuse nerve trunk inflammation may also lead to migraines, fibromyalgia, peripheral neuropathy, and/or an inability to respond to traditional medical intervention for various maladies.

Conventional tools for food allergy detection rely on antibody testing, which may provide inconsistent readings depending on where blood tested for antibodies is drawn. The gold standard of food allergy detection is simply a food elimination diet, which is a difficult, time consuming, and subjective process. Systems and methods in accordance with various embodiments of the present disclosure may overcome one or more of the aforementioned and other deficiencies experienced in conventional approaches to detecting food allergy in a human or other mammal subject. Moreover, although several embodiments are discussed with reference to food allergy, embodiments of the present disclosure can also be utilized to diagnose environmental allergies as well. In some embodiments, the allergen may be proteins or carbohydrates that are a plant product or an animal product. An allergen may be a pollen allergen, an indoor allergen, a mold allergen, an insect allergen, a food allergen, or any combination, or derivative thereof. For example, the allergen may be derived from peanut, tree nut, egg, shellfish, soy, milk, gluten, or any combination thereof.

To facilitate understanding, certain non-limiting definitions of terms are provided herein. As used herein, the term “internal body structure” and its variations include at least one of the following: a tissue, an organ, and an organ system. A tissue refers to two or more cells organized to perform one or more functions. Different types of tissue include a nervous tissue, an epithelial tissue, a connective tissue, and a muscle tissue. Examples of a nervous tissue include brain tissue, spinal cord tissue, a nerve, etc. Examples of an epithelial tissue include skin tissue, gastrointestinal (GI) tract lining, etc. Examples of a connective tissue include an internal body fluid (e.g., blood, plasma, lymph, any combination thereof, etc.), a cartilage, a bone, a ligament, a tendon, adipose tissue or fat tissue, etc. Examples of a muscle tissue include skeletal muscle, cardiac muscle, smooth muscle, etc. An organ comprises two or more types of tissue organized to perform one or more functions. Examples of an organ include a heart, a lung, a stomach, a kidney, skin, a liver, etc. An organ system includes: (i) at least two organs working together to perform one or more functions; or (ii) at least one organ and at least one tissue working together to perform one or more functions. One example of an organ system is a vascular system, which may comprise a blood vessel, an internal body fluid, and at least one organ or tissue. Another example of an organ system is a cardiovascular system, which may comprise a heart, a blood vessel, and an internal body fluid. Other examples of an organ system include a lymphatic system, a digestive system, an endocrine system, an integumentary system, a muscular system, a nervous system, a reproductive system, a respiratory system, a skeletal system, a urinary system, an immune system, a circulatory system, etc.

As used herein, the terms “circulatory system,” “circulation system,” and their variations refer to an organ system comprising a heart, at least one lung, at least one internal body fluid, and at least one vessel (e.g., a blood vessel, a lymph vessel, etc.) that functions to transport at least one internal body fluid between tissues and organs in a body. There are many types of circulation by a circulatory system. Examples include: (i) pulmonary circulation; (ii) systemic circulation; and (iii) coronary circulation. Pulmonary circulation includes transport of an internal body fluid between the heart and the lungs (e.g., from the heart, to the lungs, and back to the heart again, etc.). Systemic circulation includes transport of an internal body fluid between tissues and organs, except the heart and lungs. Coronary circulation includes transport of an internal body fluid through the tissues of the heart.

As used herein, the terms “neurovascular,” “NV,” and their variations refer to one or more internal body structures associated with a nervous system and vascular system. Examples of such structures include a blood vessel and a nerve that are coupled or connected to each other.

As used herein, the terms “arteriovenous,” “AV,” and their variations refer to a relation between an artery and a vein. For example, an AV abnormality comprises a connection or coupling between an artery and a vein, without a capillary.

As used herein, the terms “arteriovenous shunting,” “AV shunting,” “shunting,” and their variations refer to an abnormal circulation process that includes repeating a first type of circulation instead of proceeding to a second type of circulation. For example, a normal circulation process includes performing a pulmonary circulation and then a systemic circulation. With regard to this example, AV shunting occurs when the pulmonary circulation is repeated instead of moving on to the systemic circulation. Consequently, the AV shunting creates an abnormal circulation process. As used herein, the terms “arteriovenous shunt,” “AV shunt,” “shunt,” and their variations refer to the passing of an internal body structure, such as a fluid, between an artery and a vein, without the internal body structure passing through a capillary.

As used herein, the term “sonogram” and its variations refer to an image (e.g., a sound image, etc.) generated using ultrasound (US). For example, producing a sonogram includes directing US to a body part, receiving one or more echoes reflected off an internal body structure below the body part, and processing the echoes to generate a sonogram describing the internal body structure or a characteristic of the internal body structure. As used herein, the terms “ultrasound,” “US,” and their variations refer to one or more sound waves with high frequencies (e.g., a frequency equal to or greater than 20 kilohertz (KHz), etc.). US has many different applications. An exemplary application of US—referred to as diagnostic US or medical US—includes directing US to a body part to generate a sonogram describing an internal body structure below the body part or a characteristic of the internal body structure for a diagnosis within a medical context (e.g., a medical diagnosis, etc.). Examples of a diagnostic US include, but are not limited to, a Doppler US, a duplex US, and any other US or combination of USs that can create a sonogram describing an internal body fluid, a characteristic of the internal body fluid, an internal body structure associated with the internal body fluid, a characteristic of the internal body structure associated with the internal body fluid, or any combination thereof.

As used herein, “neurovascular ultrasound analysis,” “NV ultrasound analysis,” “neurovascular US analysis,” “NUA,” and its variations refer to one or more embodiments of one or more techniques of processing NV information that is based on an application of US to a subject to detect one or more abnormalities or disease states in a subject. One embodiment of NUA disclosed herein includes applying US to a body part of a subject following exposure of the subject to a potential allergen (e.g., a food substance, an environmental compound), receiving first information in response to applying the US, generating second information describing an internal body fluid under the body part or a characteristic thereof based on processing the first information generated from applying the US to the body part, generating third information describing a change in a circulatory system of the subject, and determining that the subject is allergic to the potential allergen based on processing the third information. One embodiment of NUA includes using an application of US to a body part of a subject, following exposure of the subject to a potential allergen, to determine: (i) a change in an amount of anterograde circulation; (ii) a quality of circulation between one or more arteries and one or more nerves; (iii) a change in neurovascular circulation; or (iv) any combination thereof. Other embodiments are evident from the description provided herein and the accompanying figures.

As used herein, a “subject” refers an animal, such as a mammal, including a primate (such as a human, a non-human primate, e.g., a monkey) and a non-primate (such as a mouse). In some aspects of the present disclosure, the subject is a human. In some aspects, the subject is a pediatric subject, such as a neonate, an infant, or a child. In other aspects, the subject is an adult subject.

As used in the present disclosure (e.g., some or all of the detailed description, one or more of the claims below, etc.), the phrase “at least one of A, B, or C” includes A alone, B alone, C alone, a combination of A and B, a combination of B and C, a combination of A and C, and a combination of A, B, and C. That is, the phrase “at least one of A, B, or C” means A, B, C, or any combination thereof such that one or more of a group of elements consisting of A, B and C, and should not be interpreted as requiring at least one of each of the listed elements A, B and C, regardless of whether A, B and C are related as categories or otherwise. Furthermore, the use of the article “a” or “the” in introducing an element should not be interpreted as being exclusive of a plurality of elements. Also, the recitation of “A, B and/or C” is equal to “at least one of A, B or C.” The use of “a” refers to “one or more” or “at least one” in the present disclosure. For example, “a module” refers to “one or more modules” or “at least one module.”

In some aspects, computer-implemented methods, systems, and apparatuses for conducting neurovascular ultrasound analysis for allergy testing (NUA) are provided. The NUA of the present disclosure includes an outcome-based tool, which evaluates a subject's response to an allergen, and may determine and present any changes in circulation as a consequence of exposure to the allergen. The NUA can involve testing any suitable number of sites of a subject with an ultrasound pulse Doppler test. In certain embodiments, eight sites of the subject are tested to provide efficient and reliable allergy detection. The ultrasound measurements, such as volume flow and pulsatility index for anterograde and retrograde circulation, may be recorded both before and after a subject is exposed to a potential allergen. That is, a baseline may be established at each individual area by measuring and recording the ultrasound measurement for the respective area. These ultrasound measurements are then evaluated by a controller that analyzes each part of an associated wave form and outputs an indication of an allergic reaction to the potential allergen. The present embodiments may also provide a neurovascular index (NVI), which is a tool designed to provide a quantifiable value to the quality of neurovascular circulation around a nerve. In addition, the NVI provides a user with the ability to zoom out (e.g., increase evaluation scope) and evaluate circulation of the entire neurovascular system, or zoom in (e.g., decrease evaluation scope) and examine the circulation around a particular cutaneous nerve, such as a small fiber. Furthermore, the NVI allows a user to compare the circulation through the system and provide an estimate as to the origin of a neurovascular challenge to a particular nerve. Indeed, the NVI can differentiate and quantify whether there is an autonomic challenge to the neurovascular system, where there is a neurovascular challenge to the autonomic nerves, and/or if there is a vascular bias from one extremity of the subject verses another.

In an embodiment, the NUA method starts with positioning an ultrasound device to evaluate a single area or site and gather two measurements therefrom. The measurements at that site include a pulsatility index and a volume flow. Both anterograde and retrograde versions of the pulsatility index and the volume flow are assessed. The ultrasound may generally function as a radar, which inspects the impact of structures on sound waves (instead of exact images), and presents the result of the impact and control of the structure under examination. When the ultrasound device is inspecting an artery, four individual attributes may be determined in response to the pulsed Doppler wave form being assessed via the NVI, including: (i) the level of capillary resistance below a transducer (e.g., probe) of the ultrasound device, (ii) the level of neural control of the smooth muscle below the transducer, (iii) the arterial control above the transducer, and (iv) the capillary resistance above the transducer. These four measurements, evaluated individually and/or in any suitable combination, indicate the quality of the neurovascular circulation to a nerve immediately below the transducer and associated with the inspected artery.

An increase in the capillary resistance below the transducer results in a change in the pulsatility and volume flow of the wave form of the inspected artery in a distinct way, and the NVI evaluates the wave form to associate it with a specific number that is unique for that wave form. Similarly, when sympathetic control of arterioles in the neurovascular circulation below the ultrasound device have been impaired by a lack of circulation, the NVI quantifies changes to that specific wave form. These quantification of changes to the Doppler wave form are based on observations with pre/post exposure to potential allergens and the development herein of a novel model for the NVI (e.g., NVI model), which evaluates and provides an assessment based on the close synergistic relationship between the macrovascular system and the microvascular system. Indeed, the four forces that develop or impact the pulsed Doppler wave form are derived from the relationship between the microvascular circulation and the major vessels. For one force to be accentuated and another to be reduced is defined as a deficiency likely to create ischemia or reduced blood flow. This deficiency with an imbalance between macrovascular and microvascular systems will be revealed in the pulsed Doppler wave form and quantified in the NVI utilizing the NVI model. These types of imbalances may create neural vascular congestion or even ischemia. The NVI and its associated NVI model have been developed to capture the number of deficiencies and create a percent deficit that is indicative of an allergic reaction. As such, the present embodiments provide quantification of allergic responses, which can be used to definitely confirm or disprove specific allergies for a subject.

Currently, more than fifty bodily sites have been assessed to populate the NVI model and develop the systems and methods disclosed herein. For example, the bodily sites include the cerebral vessels. The bodily sites also include the larger vessels in the body including the aorta, external and internal iliac, etc. The larger vessels may be assessed with either a vascular probe or a visceral probe of the ultrasound device, while the smaller vessels are measured with the musculoskeletal probe of the ultrasound device. In certain embodiments, the measurements are taken in the proximal and distal arm (proximal radius and ulna areas and distal radius and ulna areas). The measurements may also be taken in both legs (proximal tibia and fibula region, distal tibia and fibula region), 8-10 different regions in the feet, more than four areas in the hands, upper arms, common and deep peroneal, popliteal arteries, and so forth. As discussed below, certain embodiments of the present disclosure evaluate 10 bodily sites or areas for enhanced allergy diagnosis accuracy and reliability.

Based on the probe detecting the circulation of a vessel, a pulsed image is obtained and stored (e.g., “frozen”) in a controller communicatively coupled to the probe. A user operating the probe may then measure the volume flow in the vessel (both small and large) in both anterograde and retrograde and, further, measure the pulsatility index in the vessel (both small and large) in both anterograde and retrograde. The measurements are stored within the controller as the baseline for the subject. After exposure of the subject to the potential allergen, such as consumption of a controlled amount of a food substance by the subject or contact of a controlled amount of an environmental allergen, the foregoing measurements are taken again and compared against the baseline for the subject. The deficit for each measurement is calculated and all deficits are totaled. In certain embodiments, instead of developing an individual baseline, the deficits are calculated by comparing the post-allergen measurements to a fully functioning microvascular system around the nerve. The readings are taken and compared to an ideal ratio for each individual reading. The deficits are evaluated next to deficits with respect to other nerves, such as right and left upper extremity large fiber, right and left lower extremity large fiber, right and left upper extremity small fiber, and right and left lower extremity small fiber. Also, the deficits can be divided into right and left visceral, NR and central aorta, right and left spinal track regions, and so forth.

In some aspects of the present disclosure, a NUA system for determining a presence of an allergic reaction in a subject is provided. The NUA system includes a US probe configured to transmit a US wave to an area of the subject. The area includes an internal body structure. The NUA system includes a processor communicatively coupled to the US probe. The processor is configured to execute instructions to receive, via the US probe, a first information describing a reflection of the US wave from the area, generate a second information describing the internal body structure or a characteristic thereof based on processing the first information, generate a third information describing a change in a circulatory system of the subject based on processing the second information, determine the presence of the allergic reaction to a potential allergen in the subject based on the third information, and output, via a user interface communicatively coupled to the processor, a fourth information indicative of the presence of the allergic reaction.

In some embodiments, the third information includes one or more of fifth information describing a change in an immune response associated with the circulatory system, sixth information describing an amount of compensation on the circulatory system, and seventh information describing a change in vascular circulation by the circulatory system. In some embodiments, the processor is configured to execute the instructions to generate the second information by determining, for an extremity associated with the area, at least one of an anterograde pulsatility index, an anterograde volume flow, a retrograde pulsatility index, and a retrograde volume flow, and determining a neurovascular index for the extremity composed of the at least one of the anterograde pulsatility index, the anterograde volume flow, the retrograde pulsatility index, and the retrograde volume flow. In some embodiments, the processor is configured to execute the instructions to determine an arteriovenous (AV) shunting score for the extremity based on the neurovascular index and a predetermined extremity score. In some embodiments, the processor is configured to execute the instructions to determine a cumulative anterograde volume flow score based on a sum of respective anterograde volume flows of the extremity and at least one additional extremity of the subject. In some embodiments, the processor is configured to execute the instructions to generate the third information by determining a decrease in the cumulative anterograde volume flow score and generating fifth information describing a change in an immune response associated with the circulatory system based on the decrease in the cumulative anterograde volume flow score.

In some embodiments, the processor is configured to execute the instructions to generate the third information by determining an increase in an amount of compensation on the circulatory system and generating sixth information describing the amount of compensation on the circulatory system based on the increase in the amount of compensation on the circulatory system. In some embodiments, the processor is configured to execute the instructions to generate the third information by determining a decrease in an amount of the internal body structure or the characteristic thereof circulating through the circulatory system and generating seventh information describing a change in vascular circulation by the circulatory system based on the decrease in the amount of the internal body structure or the characteristic thereof circulating through the circulatory system. In some embodiments, the processor is configured to output the fourth information indicative of the presence of the allergic reaction following exposure of the subject to the potential allergen. In some embodiments, the processor is configured to output a pre-exposure fourth information indicative of the presence of the allergic reaction prior to exposure of the subject to the potential allergen to establish baseline measurements and output the fourth information indicative of the presence of the allergic reaction following exposure of the subject to the potential allergen and based on the baseline measurements and post-exposure measurements. In some embodiments, the baseline measurements are measurements acquired or determined prior to exposure of the subject to the allergen and may be reflective of the normal state of the subject.

In some embodiments, the NUA is based on the immune activation in response to an allergen and cardiac compensation following the immune activation. Immune response includes a histamine release and cardiac compensation, which create wave form changes (as analyzed through NVI method) and increase in volume flow in response to exposure to the allergen. In some embodiments, the positive criteria for allergy detection with respect to a baseline-generated control include determining: (1) a percent difference between a current value and a corresponding control value increases by greater than 1.5 or decreases below 0.85; (2) UE increases by greater than 1.25 and LE decreases by more than 0.8; (3) AV shunting changes from UE to LE dominance; (4) total AV shunting changes greater than 0.15; or (5) any combination thereof.

In some embodiments for detection of a food allergy, the NUA of the present disclosure includes the following steps: assessing macro/micro relationship after introducing a food, assessing the immune response, and assessing the cardio compensation for immune response. As should be understood with respect to the discussions above and below, the methods, systems, and apparatuses of the present disclosure can be used in clinic settings to test food allergy and identify allergenic/non-allergenic food in subjects. These methods, systems, and apparatuses provide noticeable benefits over prior antibody detection or food elimination diets, including non-subjectivity, ease, and reduced invasiveness of allergy detection.

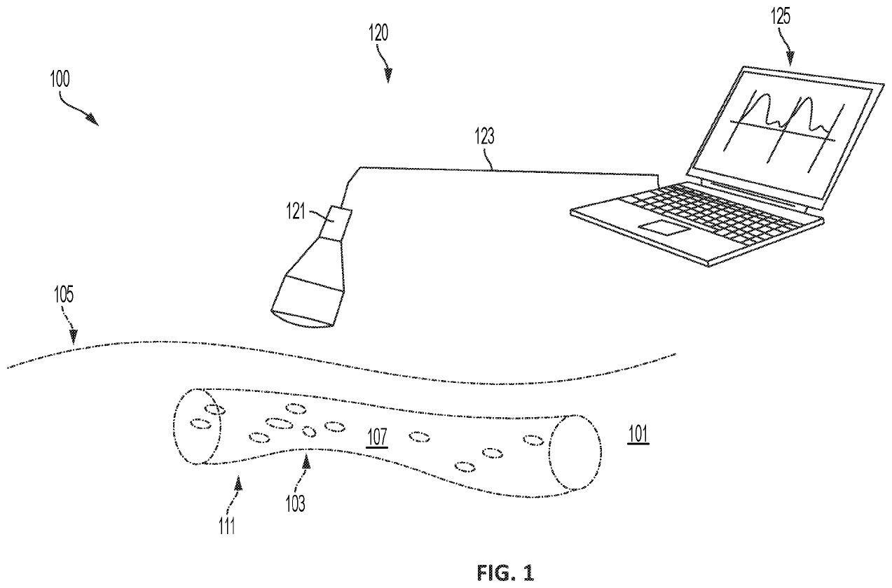

In an example, a computer-implemented system is disclosed for neurovascular (NV) ultrasound (US) analysis (NUA) for an allergy detection. FIG. 1 illustrates an example of computer-implemented NUA system 100, in accordance with aspects of the present disclosure. As will be understood, the NUA system 100 may detect an allergy based on monitoring a series of controlled physical changes to at least one area 101 of at least one extremity of a subject. The extremity can be an upper extremity or a lower extremity, such as a hand, arm, leg, ankle, or foot, while in still other examples, various other portions of the subject may be targeted, such as the neck or the back. The area 101 of the subject includes an internal body structure 103 disposed beneath a surface 105 (e.g., epidermis) and at least partially within the extremity of the subject. In certain embodiments, the internal body structure 103 includes an artery or a vessel through which a fluid 107 flows. The internal body structure 103 is associated with a circulatory system 111, which may directly or indirectly control flow of the fluid 107 within the internal body structure 103.

The NUA system 100 of the present embodiment also includes a US device 120. The US device 120 includes a US probe 121, which is positioned on or in the area 101 of the subject. As discussed above, the US probe 121 may be used to establish a baseline response of the subject and may be used following an exposure of a potential allergen to the subject to analyze any allergic response to the allergen. The US probe 121 of the present embodiment is communicatively coupled via a cable 123 to one or more computers, hereinafter referred to as a controller 125. In other embodiments, the US probe 121 and the controller 125 are wirelessly communicatively coupled.

As illustrated, the US device 120 emits a US wave via the US probe 107 into the area 101, such as based on an instruction from the controller 125. From the US probe 107, the controller 125 of the NUA system 100 receives first information describing a reflection of the US wave from the area 101. The NUA system 100 generates second information describing the internal body structure 103 or a characteristic of the internal body structure 103 based on processing the first information, such as via the controller 125. The controller 125 of the NUA system 100 generates third information describing a change in the circulatory system 111 of the subject based on processing the second information. The NUA system 100 may therefore detect an allergy based on processing the third information and generate fourth information describing the detecting of the allergy for output to a user of the NUA system 100. Further non-limiting examples regarding the information generated, processed, and used by the NUA system 100 are provided below, followed by certain examples of components and configurations of the NUA system 100. As will be understood, the NVI presents and analyzes bodily inflammation information for allergy detection, where increased inflammation readings after allergen exposure indicate an allergic reaction. Further, if an allergic response is detected in the subject, then the human subject may be administered with an anti-allergy medication to alleviate any symptom associated with the detected allergy. In some embodiments, the allergy medication may be an antihistamine, a corticosteroid, a bronchodilator, a mast cell stabilizer, a leukotriene inhibitor, a serine lung protease inhibitor, or any combination, or derivative thereof. In some embodiments, the antihistamine may be a H1-antihistamine, H2-antihistamine, H3-antihistamine, H4-antihistamine, or any combination thereof.

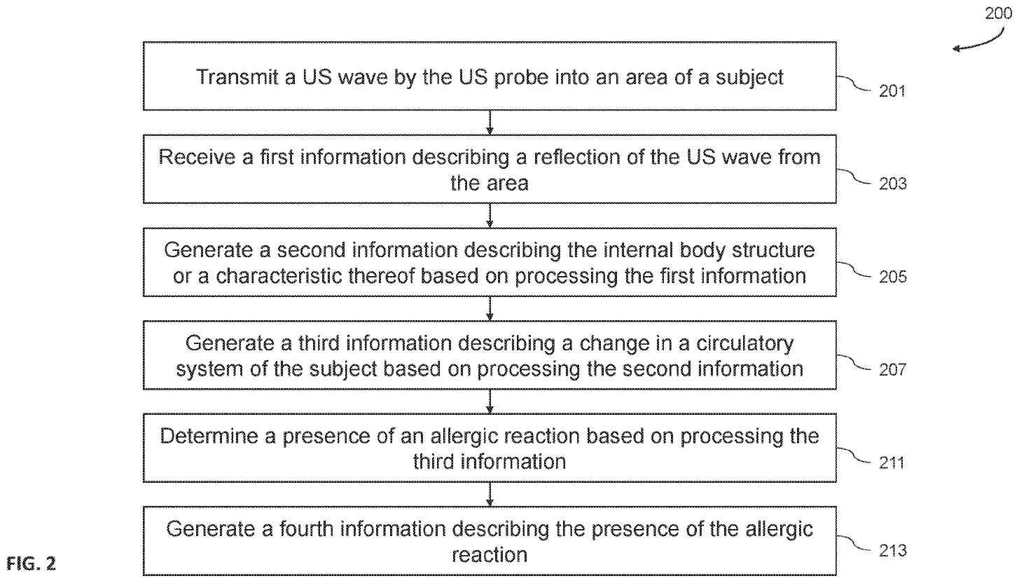

FIG. 2 is a block diagram illustrating an exemplary computer-implemented method 200 for detecting food allergy in a subject via the NUA system disclosed herein. In certain embodiments, the method 200 is performed by the US probe 107 and/or a processor of the controller 125 of FIG. 1. In FIG. 2, the method 200 begins at block 201 with transmitting a US wave from a US probe into an area of a subject. In certain embodiments, the US probe transmits the US wave in response to one or more instructions from a controller. For each tested area, the US probe may be positioned on or in the area of a subject by a user or a healthcare provider. The healthcare provider may position the US probe subsequent to or following a consumption of food by the subject to analyze an allergic response to the allergen or food. In certain embodiments, the allergic response to the allergen may be evaluated a predetermined time period after the subject is exposed to the allergen. In certain embodiments, the allergic response may be evaluated after the subject experiences a visible or perceivable reaction to the allergen. Moreover, certain NUA embodiments facilitate allergy diagnosis based on US measurements taken at ten different bodily sites or areas. In certain embodiments, the areas include four upper extremity areas and six lower extremity areas, such as two areas on a radial side of an arm, two areas on an ulnar side of the arm, three areas on a lower medial side of the leg (e.g., tibial), and three areas on a lower lateral side of the leg (e.g., peroneal). Each of the ten areas may be measured via US before allergen exposure and then again after the allergen exposure, in certain embodiments.

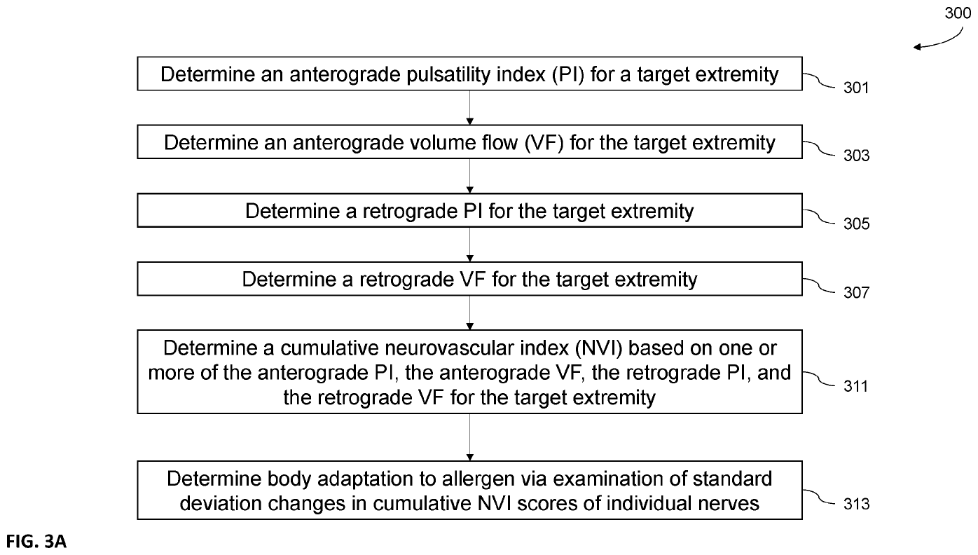

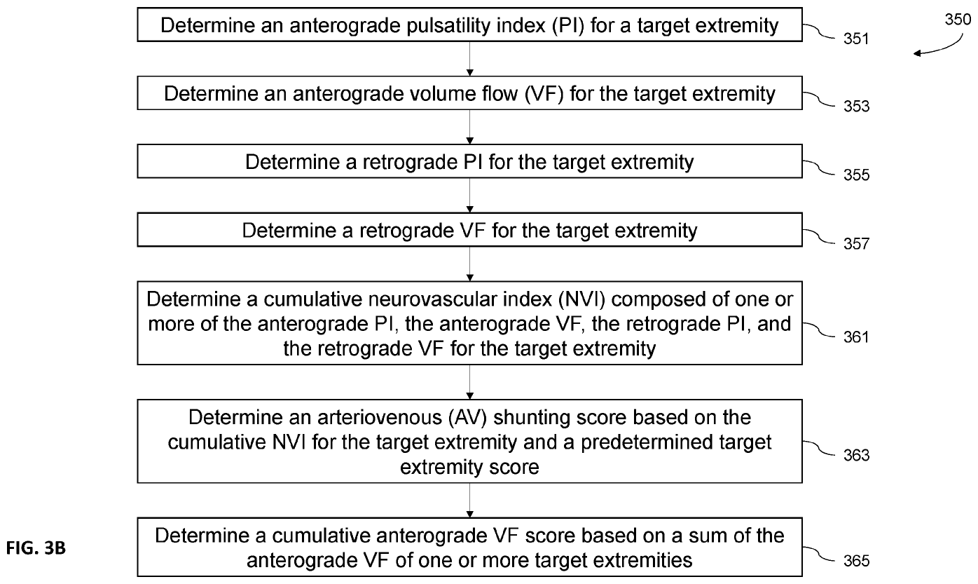

At block 203, a first information is received describing a reflection of the US wave from the area. For example, the processor may receive the first information from the US probe. In certain embodiments, the first information includes a Doppler wave form associated with the area. At block 205, a second information is generated describing the internal body structure or a characteristic thereof based on processing the first information. In certain embodiments, the processor generates the second information by analyzing metrics of the Doppler wave form associated with the area. Certain examples of generating the second information are discussed in more detail below with respect to the methods 300, 350 of FIGS. 3A, 3B. For example, the second information of certain embodiments discussed in more detail below may include an anterograde pulsatility index (PI), an anterograde volume flow (VF), a retrograde PI, a retrograde VF, an AV shunting score, and/or a cumulative anterograde VF score.

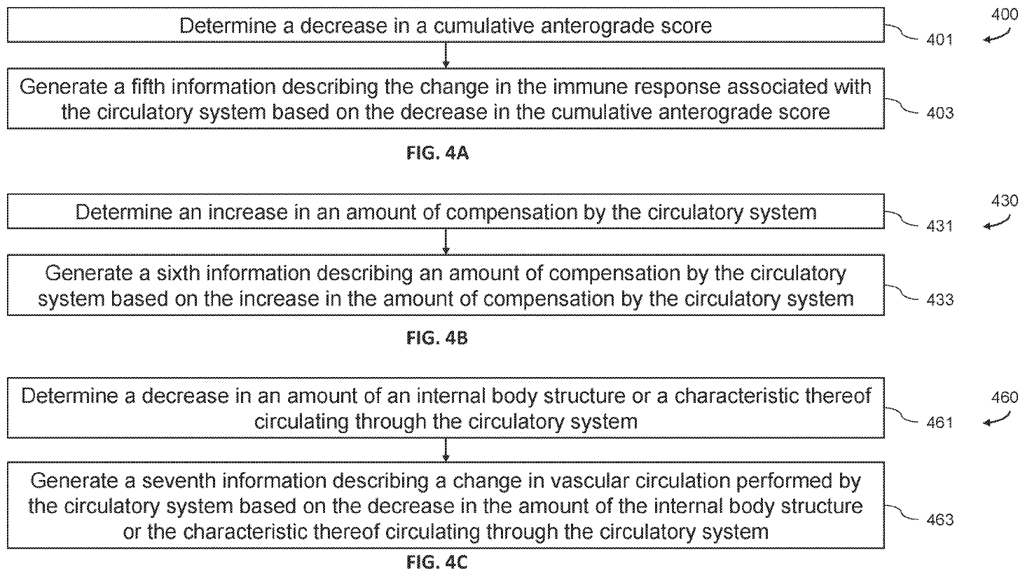

At block 207, a third information is generated describing a change in a circulatory system of the subject based on processing the second information. The processor of certain embodiments generates the third information that includes (1) a fifth information describing a change in an immune response associated with the circulatory system, (2) a sixth information describing an amount of compensation on the circulatory system, and/or (3) a seventh information describing a change in vascular circulation by the circulatory system. Certain embodiments of generating the fifth, sixth, and/or seventh information for embodiments of the third information are discussed below with respect to the methods of FIGS. 4A-4C. At block 211, a presence of an allergic reaction or a food allergy is determined (e.g., detected) based on processing the third information. For example, the processor of certain embodiments may determine that the third information is indicative of the presence of the allergic reaction in response to comparing a respective value of the third information to either one of a corresponding individualized baseline value or a generalized reference value. In certain embodiments, the processor may determine the third information is indicative of the presence of the allergic reaction in response to (1) the fifth information describing the change in the immune response being greater than an immune response threshold, (2) the sixth information describing the amount of circulatory system compensation being greater than a compensation threshold, and/or (3) the seventh information describing the change in vascular circulation being greater than a vascular circulation threshold. At block 213, a fourth information describing the presence of the allergic reaction is generated. In certain embodiments, the processor may output the fourth information via a user interface communicatively coupled to the processor via any suitable visual and/or audible alerts. In these embodiments, the NUA system desirably presents an indication of allergy to the healthcare provider and/or the subject so that properly informed isolation of the subject from corresponding allergens may be performed.

As disclosed herein, the methods for NUA facilitate determination of a degree of allergic reaction to a tested allergen. For example, the NUA may indicate the subject has no reaction, a low response, a moderate response, or a severe response to the allergen based on the quantification of the inflammation sensed in the body of the subject. Indeed, the NVI presents and analyzes bodily inflammation information for allergy detection, where increased inflammation readings after allergen exposure indicate an allergic reaction.

FIG. 3A is a block diagram illustrating an exemplary computer-implemented method 300 for generating the second information describing the internal body structure or the characteristic thereof based on processing the first information (as exemplified in block 205 of the method 200). All or a portion of the method 300 may be performed by a processor or controller of the NUA system, in certain embodiments. Moreover, the method 300 may be repeated for each suitable extremity of the subject, such as one or more of an upper left extremity, a lower left extremity, an upper right extremity, and a lower right extremity. In FIG. 3A, at block 301, an anterograde pulsatility index (PI) is determined for a target extremity of the subject. As used herein, pulsatility index is defined as the difference between a peak systolic flow velocity and a minimum diastolic flow velocity, divided by a mean velocity recorded throughout a cardiac cycle. At block 303, an anterograde volume flow (VF) is determined for the target extremity. At block 305, a retrograde PI is determined for the target extremity. At block 307, a retrograde VF is determined for the target extremity. The anterograde measurements are evaluated with respect to a forward flow direction and the retrograde measurements are evaluated with respect to a reverse flow direction, as would be understood one of ordinary skill in the art.

At block 311, a cumulative neurovascular index (NVI) for the target extremity is determined. The NVI is composed of one or more of the anterograde PI, the anterograde VF, the retrograde PI, and the retrograde VF. In certain embodiments, a threshold number of the anterograde PI, the anterograde VF, the retrograde PI, and the retrograde VF may be evaluated to further improve the accuracy and reliability of the NUA system disclosed herein. For example, the NUA system may stipulate that the threshold number is two, three, or four, in certain embodiments. In embodiments in which one or more of the anterograde PI, the anterograde VF, the retrograde PI, and the retrograde VF are not utilized for determination of the cumulative NVI, the corresponding one or more of blocks 301-307 may be omitted. In certain embodiments, the anterograde PI, the anterograde VF, the retrograde PI, and/or the retrograde VF correspond to the second information describing the internal body structure or the characteristic thereof.

In certain embodiments, the NUA assessment evaluates any waveform variability changes in response to the tested allergen that are outside of a normal range. In some of these embodiments, the normal range of NVI is 7-14. However, it should be understood that any suitable range for allergy detection based on variability changes may be established. In certain embodiments, the NUA assessment evaluates any abnormal anterograde volume flow changes, such as increases or decreases that are outside of normal variance. These volume flow changes may include: (1) an increase in anterograde volume flow indicating cardiac changes (e.g., increases) in response to inflammation, such as greater than 1.5 times control, (2) a decrease in anterograde volume flow indicating histamine release in response to the tested allergen, and/or (3) a combination of upper extremity increase and lower extremity decrease that indicates pattern changes in relationship to position of a tested area relative to the heart.

At block 313, a body adaptation to a tested allergen is determined via examination of standard deviation changes in cumulative NVI of individual nerves. Standard deviation analysis is performed to quantify certain immune responses, as discussed herein and illustrated in Example 2 below. For example, an allergy diagnosis may be confirmed in response to standard deviation analysis indicating that a difference between the baseline reading and a post-exposure reading along a nerve is statistically significant. As disclosed herein, certain individual nerves that may be evaluated include fibular nerves, tibial nerves, back nerves, and neck nerves. In certain embodiments, the standard deviation analysis or data thereof corresponds to the second information describing the internal body structure or the characteristic thereof.

FIG. 3B is a block diagram illustrating an exemplary computer-implemented method 350 for generating the second information describing the internal body structure or the characteristic thereof based on processing the first information (as exemplified in block 205 of the method 200). All or a portion of the method 350 may be performed by a processor or controller of the NUA system, in certain embodiments. Moreover, the method 350 may be repeated for each suitable extremity of the subject, such as one or more of an upper left extremity, a lower left extremity, an upper right extremity, and a lower right extremity. The method 350 may be performed in combination or in parallel with the method 300, in certain embodiments. In other embodiments, only one of the methods 300, 350 may be performed.

In FIG. 3B, at block 351, an anterograde PI is determined for a target extremity of the subject, such as discussed above at block 301 of FIG. 3A. At block 353, an anterograde VF is determined for the target extremity. At block 355, a retrograde PI is determined for the target extremity. At block 357, a retrograde VF is determined for the target extremity. At block 361, a cumulative neurovascular index (NVI) for the target extremity is determined. The NVI is composed of one or more of the anterograde PI, the anterograde VF, the retrograde PI, and the retrograde VF. These steps of FIG. 3B may correspond to the above-discussed steps of FIG. 3A, in certain embodiments.

At block 363, an arteriovenous (AV) shunting score is determined based on the cumulative NVI for the extremity and a predetermined target extremity score. At block 365, a cumulative anterograde VF score is determined based on a sum of the anterograde VF of one or more target extremities. In certain embodiments, the AV shunting score and/or the cumulative anterograde VF score also correspond to the second information describing the internal body structure or the characteristic thereof.

As noted above, the third information describing a change in a circulatory system of certain embodiments may include (1) information regarding a change in an immune response associated with the circulatory system (e.g., fifth information), (2) information regarding an amount of compensation on the circulatory system (e.g., sixth information), and/or (3) a change in vascular circulation by the circulatory system (e.g., seventh information). To facilitate understanding, FIGS. 4A-4C are provided below to discuss each of these components individually. However, one or more of the methods of FIGS. 4A-4C may be performed simultaneously or sequentially, in any suitable order. Moreover, the methods of FIGS. 4A-4C may be performed by a processor or any other suitable component of the NUA system disclosed herein.

FIG. 4A is a block diagram illustrating an exemplary computer-implemented process 400 for generating the third information describing the change in the circulatory system of the subject based on processing the second information (as exemplified in block 207 of the method 200). In FIG. 4A, at block 401, a decrease in a cumulative anterograde VF score is determined. For example, the cumulative anterograde VF score may be determined to have a first value in a first time period and a lower second value at a lower second time period. At block 403, the fifth information is generated describing the change in the immune response associated with the circulatory system based on the decrease in the cumulative anterograde VF score. In certain embodiments, the cumulative anterograde VF score and the corresponding determined decrease may be generated via block 365 of the method 350. In such embodiments, the cumulative anterograde VF score corresponds to the second information and the decrease in the cumulative anterograde VF score corresponds to the third information and/or the fifth information.

Additionally, or alternatively, FIG. 4B shows a block diagram illustrating an exemplary computer-implemented method 430 for generating the third information describing the change in the circulatory system of the subject based on processing the second information (as exemplified in block 207 of the method 200). In FIG. 4B, an increase in an amount of compensation on the circulatory system is determined at block 431. As disclosed herein, immune activation may instigate cardiac or circulatory system compensation, which generates Doppler wave form changes. The wave form changes may be detected and evaluated via the US probe and/or controller of the presently disclosed NUA system. At block 433, the sixth information is generated describing an amount of compensation on the circulatory system based on the increase in the amount of compensation on the circulatory system. In such embodiments, the Doppler wave form changes correspond to the second information and the increase in the amount of circulatory system compensation based on the Doppler wave form changes corresponds to the third information and/or the sixth information.

Additionally, or alternatively, FIG. 4C shows a block diagram illustrating an exemplary computer-implemented method 460 for generating the third information describing the change in the circulatory system of the subject based on processing the second information (as exemplified in block 207 of the method 200). In FIG. 4C, a decrease in an amount of the internal body structure or a characteristic thereof circulating through the circulatory system is determined at block 461. At block 463, the seventh information is generated describing a change in vascular circulation by the circulatory system based on the decrease in the amount of the internal body structure or the characteristic thereof circulating through the circulatory system. In such embodiments, the amount of the internal body structure or the characteristic thereof circulating through the circulatory system corresponds to the second information and the change in vascular circulation corresponds to the third information and/or the seventh information.

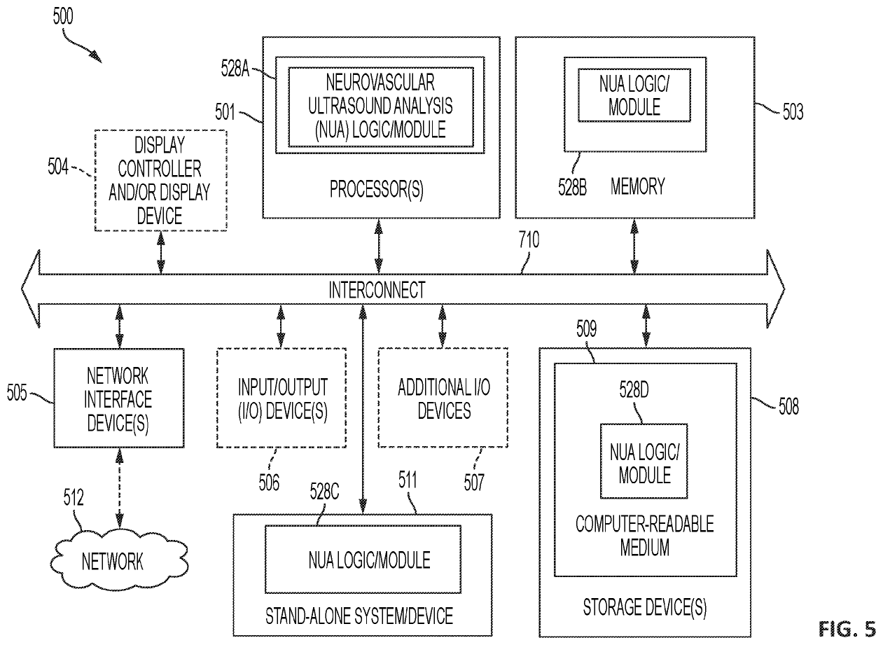

FIG. 5 is a block diagram illustrating an exemplary data processing system 500 that may be used with one or more of the described embodiments. For example, the system 500 may represent any data processing system (e.g., one or more of the systems described above performing any of the operations or methods described above in connection with FIGS. 1-4, etc.). The system 500 can include many different components. These components can be implemented as integrated circuits (ICs), portions thereof, discrete electronic devices, or other modules adapted to a circuit board such as a motherboard or add-in card of a computer system, or as components otherwise incorporated within a chassis of a computer system. Note also that system 500 is intended to show a high-level view of many, but not all, components of the computer system. Nevertheless, it is to be understood that additional components may be present in certain implementations and furthermore, different arrangements of the components shown may occur in other implementations. The system 500 may represent a desktop computer system, a laptop computer system, a tablet computer system, a server computer system, a mobile phone, a media player, a personal digital assistant (PDA), a personal communicator, a network router or hub, a wireless access point (AP) or repeater, a set-top box, or a combination thereof. Further, while only a single machine or system is illustrated, the term “machine” or “system” shall also be taken to include any collection of machines or systems that individually or jointly execute instructions to perform any of the methodologies discussed herein.

For one embodiment, the system 500 includes processor(s) 501, memory 503, network interface device(s) 505, input/output (I/O) device(s) 506, additional I/O devices 507, storage device(s) 508, stand-alone system/device 511 via a bus or an interconnect 710. The system 500 also includes a network 512. Processor(s) 501 may represent a single processor or multiple processors with a single processor core or multiple processor cores included therein. Processor(s) 501 may represent one or more general-purpose processors such as a microprocessor, a central processing unit (CPU), graphics processing unit (GPU), or the like. More particularly, processor(s) 501 may be a complex instruction set computer (CISC), a reduced instruction set computer (RISC) or a very long instruction word (VLIW) computer architecture processor, or processors implementing a combination of instruction sets. Processor(s) 501 may also be one or more special-purpose processors such as an application specific integrated circuit (ASIC), an application-specific instruction set processor (ASIP), a cellular or baseband processor, a field programmable gate array (FPGA), a digital signal processor (DSP), a physics processing unit (PPU), an image processor, an audio processor, a network processor, a graphics processor, a graphics processing unit (GPU), a network processor, a communications processor, a cryptographic processor, a co-processor, an embedded processor, a floating-point unit (FPU), or any logic that can process instructions.

Processor(s) 501, which may be a low power multi-core processor socket such as an ultra-low voltage processor, may act as a main processing unit and central hub for communication with the various components of the system. Such processor(s) can be implemented as one or more system-on-chip (SoC) integrated circuits (ICs). A neurovascular ultrasound (NUA) logic/module 528A may reside, completely or at least partially, within processor(s) 501. In one embodiment, the NUA logic/module enables the processor(s) 501 to perform any or all of the operations or methods described above in connection with FIG. 1-4. Additionally, or alternatively, the processor(s) 501 may be configured to execute instructions for implementing or performing the subject matter (e.g., operations, methodologies, etc.) discussed herein.

The system 500 may further include a graphics interface that communicates with optional graphics subsystem, which may include a display controller, a graphics processing unit (GPU), and/or a display device. Processor(s) 501 may communicate with memory 503, which in one embodiment can be implemented via multiple memory devices to provide for a given amount of system memory. Memory 503 may include one or more volatile storage (or memory) devices such as random access memory (RAM), dynamic RAM (DRAM), synchronous DRAM (SDRAM), static RAM (SRAM), or other types of storage devices. Memory 503 may store information including sequences of instructions that are executed by processor(s) 501 or any other device. For example, executable code and/or data from a variety of operating systems, device drivers, firmware (e.g., input output basic system or BIOS), and/or applications can be loaded in memory 503 and executed by processor(s) 501. An operating system can be any kind of operating system. A NUA logic/module 528B may also reside, completely or at least partially, within memory 503.

For one embodiment, the memory 503 includes a NUA logic/module as executable instructions. For another embodiment, when the instructions represented by DAM logic/module are executed by the processor(s) 501, the instructions cause the processor(s) 501 to perform any, all, or some of the operations or methods described above in connection with FIGS. 1-4.

The system 500 may further include I/O devices such as devices 506, including network interface device(s) 505, optional input device(s), and other optional I/O device(s) 507. Network interface device 505 may include a wired or wireless transceiver and/or a network interface card (NIC). The wireless transceiver may be a WiFi transceiver, an infrared transceiver, a Bluetooth transceiver, a WiMax transceiver, a wireless cellular telephony transceiver, a satellite transceiver (e.g., a global positioning system (GPS) transceiver), or other radio frequency (RF) transceivers, or a combination thereof. The NIC may be an Ethernet card.

Input device(s) may include a mouse, a touch pad, a touch sensitive screen (which may be integrated with display device, a pointer device such as a stylus, and/or a keyboard (e.g., a physical keyboard or a virtual keyboard displayed as part of a touch sensitive screen). For example, input device may include a touch screen controller coupled to a touch screen. The touch screen and touch screen controller can, for example, detect contact and movement or a break thereof using one or more touch sensitivity technologies, including but not limited to capacitive, resistive, infrared, and surface acoustic wave technologies, as well as other proximity sensor arrays or other elements for determining one or more points of contact with the touch screen.

I/O devices 506 may include an audio device. An audio device may include a speaker and/or a microphone to facilitate voice-enabled functions, such as voice recognition, voice replication, digital recording, and/or telephony functions. Other I/O devices 507 may include universal serial bus (USB) port(s), parallel port(s), serial port(s), a printer, a network interface, a bus bridge (e.g., a PCI-PCI bridge), sensor(s) (e.g., a motion sensor such as an accelerometer, gyroscope, a magnetometer, a light sensor, compass, a proximity sensor, etc.), or a combination thereof. Device(s) may further include an imaging processing subsystem (e.g., a camera), which may include an optical sensor, such as a charged coupled device (CCD) or a complementary metal-oxide semiconductor (CMOS) optical sensor, utilized to facilitate camera functions, such as recording photographs and video clips. Certain sensors may be coupled to interconnect 710 via a sensor hub (not shown), while other devices such as a keyboard or thermal sensor may be controlled by an embedded controller (not shown), dependent upon the specific configuration or design of system 500.

To provide for persistent storage for information such as data, applications, one or more operating systems and so forth, a mass storage device or devices (not shown) may also be coupled to processor(s) 501. For various embodiments, to enable a thinner and lighter system design as well as to improve system responsiveness, this mass storage may be implemented via a solid state device (SSD). However, in other embodiments, the mass storage may primarily be implemented using a hard disk drive (HDD) with a smaller amount of SSD storage to act as a SSD cache to enable non-volatile storage of context state and other such information during power down events so that a fast power up can occur on re-initiation of system activities. In addition, a flash device may be coupled to processor(s) 501, e.g., via a serial optional peripheral interface (SPI). This flash device may provide for non-volatile storage of system software, including a basic input/output software (BIOS) and other firmware.

A NUA logic/module 528C may be part of a specialized stand-alone computing system/device 511 that is formed from hardware, software, or a combination thereof. For one embodiment, the NUA logic/module 528C performs any, all, or some of the operations or methods described above in connection with FIGS. 1-4.

Storage device 508 may include computer-accessible storage medium 509 (also known as a machine-readable storage medium or a computer-readable medium) on which is stored one or more sets of instructions or software—e.g., a NUA logic/module 528D.

For one embodiment, the instruction(s) or software stored on storage medium 508 embody one or more methodologies or functions described above in connection with FIGS. 1-4. For another embodiment, the storage device 508 includes a NUA logic/module as executable instructions. When the instructions represented by a NUA logic/module are executed by the processor(s) 501, the instructions cause the system 500 to perform any, all, or some of the operations or methods described above in connection with FIGS. 1-4.

Computer-readable storage medium 509 can store some or all of the software functionalities of a NUA logic/module described above persistently. While computer-readable storage medium 509 is shown in an exemplary embodiment to be a single medium, the term “computer-readable storage medium” should be taken to include a single medium or multiple media (e.g., a centralized or distributed database, and/or associated caches and servers) that store the one or more sets of instructions. The terms “computer-readable storage medium” shall also be taken to include any medium that is capable of storing or encoding a set of instructions for execution by the system 500 and that cause the system 500 to perform any one or more of the disclosed methodologies. The term “computer-readable storage medium” shall accordingly be taken to include, but not be limited to, solid-state memories, and optical and magnetic media, or any other non-transitory machine-readable medium.

Note that while system 500 is illustrated with various components of a data processing system, it is not intended to represent any particular architecture or manner of interconnecting the components; as such, details are not germane to the embodiments described herein. It will also be appreciated that network computers, handheld computers, mobile phones, servers, and/or other data processing systems, which have fewer components or perhaps more components, may also be used with the embodiments described herein.

In some aspects, the present disclosure provides a NUA system for determining a presence of an allergic reaction in a subject. The neurovascular US analysis system includes a US probe configured to transmit a US wave to an area of the subject. The area includes an internal body structure. The neurovascular US analysis system includes a processor communicatively coupled to the US probe. The processor is configured to execute instructions to receive, via the US probe, a first information describing a reflection of the US wave from the area, generate a second information describing the internal body structure or a characteristic thereof based on processing the first information, generate a third information describing a change in a circulatory system of the subject based on processing the second information, determine the presence of the allergic reaction to a potential allergen in the subject based on the third information, and output, via a user interface communicatively coupled to the processor, a fourth information indicative of the presence of the allergic reaction. In some embodiments, the third information includes one or more of fifth information describing a change in an immune response associated with the circulatory system, sixth information describing an amount of compensation on the circulatory system, and seventh information describing a change in vascular circulation by the circulatory system.

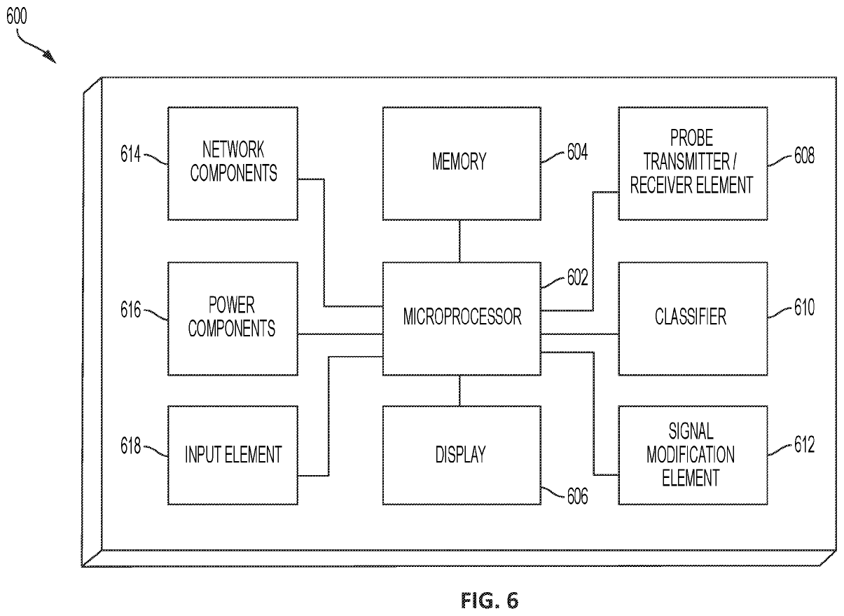

FIG. 6 illustrates an example device 600 providing a computer-implemented method for neurovascular ultrasound analysis (NUA) in a system, in accordance with aspects of this disclosure. Particularly, an example set of components 602-618 are provided in the device 600. As such, device 600 may be the computer device in the example of FIG. 1, according to an embodiment. In an example, the device 600 may be an ultrasound machine with developed capabilities based on the present disclosure. For example, the developed capabilities enable the ultrasound machine to identify and process discriminant features from each of the first, second, third, and fourth signals referenced in prior examples. As such, the ultrasound machine 600 includes instructions in memory 604 for the microprocessor 602, classifier 610, and signal modification element 612 to generate or provide discriminant features from each of the first, second, and third signals. The discriminant features in each of the first, second, and third signals may include velocity values clustering in a two-dimensional plane to support a determination that a neuropathy issue is identified and is being managed.

The illustrated example device 600 includes at least one main microprocessor 602 for executing instructions stored in physical memory 604 on the device, such as dynamic random-access memory (DRAM) or flash memory, among other such options. As would be apparent to one of ordinary skill in the art, the device 600 can include many types of memory, data storage, or computer-readable media as well, such as a hard drive or solid state memory functioning as data storage or memory 604 for the device. Application instructions for execution by the at least one microprocessor 602 can be stored by an extra data storage (separate from memory 604), that is then loaded into memory 604 as needed for operation of the device 600. The microprocessor 602 can have internal memory, as well, to be used in some embodiments for temporarily storing data and instructions for processing. The device 600 can also support removable memory (as part of or separate from memory 604) useful for sharing information with other devices. The device may also include one or more power components 616 for powering the device. The power components can include, for example, a battery compartment for powering the device using a rechargeable battery, an internal power supply, or a port for receiving external power, among other such options, as will be readily understood by one having ordinary skill in the art.

The computing device may include, or may be in communication with, at least one type of display element 606, such as a touch screen, organic light emitting diode (OLED), or liquid crystal display (LCD). Some devices may include multiple display elements, and may also include LEDs, projectors, and the like. The device can include at least one communication or networking component 614, and may enable transmission and receipt of various types of data or other electronic communications. The communications may occur over any appropriate type of network, such as the Internet, an intranet, a local area network (LAN), a 5G or other cellular network, or a Wi-Fi network, or can utilize transmission protocols such as BLUETOOTH® or NFC, among others. The device can include at least input element 618 capable of receiving input from a user or other source. This input device can include, for example, a button, dial, slider, touch pad, wheel, joystick, keyboard, mouse, trackball, camera, microphone, keypad, or other such device or component. Various devices can also be connected by wireless or other such links as well, in some embodiments. In some embodiments, a device might be controlled through a combination of visual and audio commands, or gestures, such that a user can control the device without having to be in contact with the device or a physical input mechanism. In addition, the device 600 can include a probe transmitter/receiver element 608 for providing and receiving signals, such as Doppler audio signals. In an operative example, Doppler signals also may be converted to audio signals, with higher velocities of blood flow providing high-pitched sounds, while lower velocities provide low-pitched sounds. The classifier 610 may be a neural network classifier for classifying discriminant features of the audio signals—or directly from the Doppler audio sounds. The discriminant features may be Fourier transformed audio signals or non-transformed velocity values as presented from the audio signals.

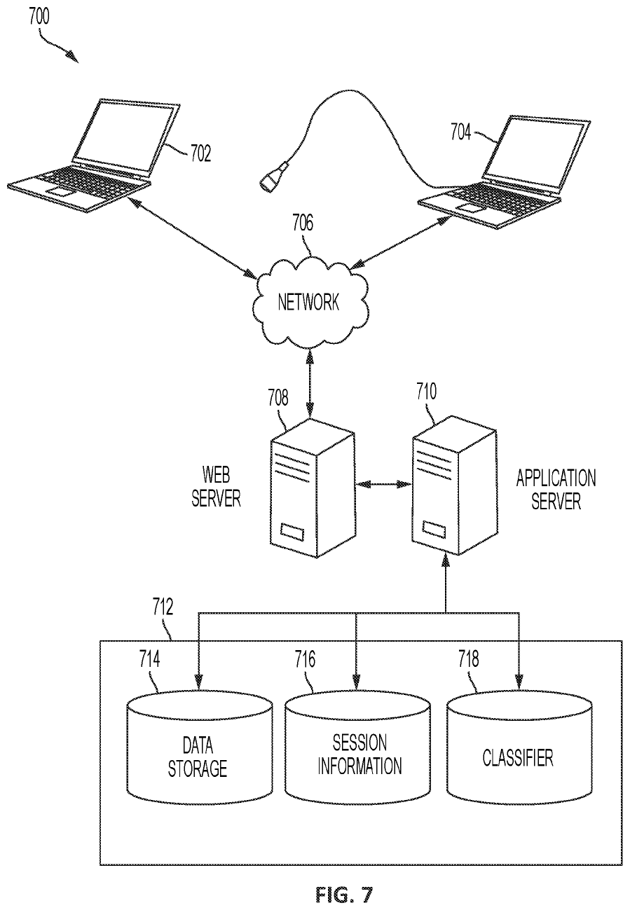

Much of the functionality utilized with various embodiments may be operated in a computing environment that may be operated by, or on behalf of, a healthcare provider or entity. Alternatively, or in addition, there may be dedicated computing resources or resources allocated as part of a cloud environment. FIG. 7 illustrates an example network architecture or environment 700 used in a system for computer-implemented method for neurovascular ultrasound analysis (NUA) to detect a food allergy, in accordance with aspects of this disclosure. In addition, the example network architecture or environment 700 may be used to share the NUA detection process or to control the NUA detection process remotely. The resources can utilize any of a number of operating systems and applications, and can include any number of workstations or servers 702, 704. Various embodiments may utilize at least one cloud or internet-based network 706 for supporting communications using any of a variety of commercially-available protocols, such as TCP/IP or FTP, among others. Other example networks 706 include, for example, a local area network, a wide-area network, a virtual private network, the Internet, an intranet, and various combinations thereof. The servers 712 may be used to host an offering such as portions of the classifier 610. This allows retraining of the classifier to provide a testing neural network or a training neural network for improving system accuracy. In an example, once the wave forms are identified for a subject with a detected allergy, the wave form values or points are stored to train the network. Once NUA detection is administered, the wave form improvements are provided in the form of new values or points that can be used to train a second network. Then, for future human subjects, the human subject is tested against the two networks to determine where the wave form obtained from each future human subject classifies. When the classification is indicative of normal blood flow patterns, then a condition of the human subject is determined as non-allergic. Further, if the classification is indicative of an allergic response in the subject, then the human subject may be administered with an anti-allergy medication to alleviate any symptom associated with the detected allergy. In some embodiments, the allergy medication may be an antihistamine, a corticosteroid, a bronchodilator, a mast cell stabilizer, a leukotriene inhibitor, a serine lung protease inhibitor, or any combination, or derivative thereof. In some embodiments, the antihistamine may be a H1-antihistamine, H2-antihistamine, H3-antihistamine, H4-antihistamine, or any combination thereof.

As the neural network process is a complex data intensive process, the classifier or portions of the classifier 718 may be operational from servers 712. Data for each session of the NUA detection may be encrypted and stored in data storage 714 with anonymity but for indications of severity of the condition. The session information 716 is useful for ongoing NUA detections, and the data may be moved from the session information 716 to the data storage 714 after the session is complete. The data then may be moved to the classifier to improve the classifier 718. In an example, apart from neural networks, support vector machines (SVM) or k-nearest neighbor algorithms may be used. As with the case of the neural network, pitch or other features, including Fourier transforms and linear predictive coefficients, may be extracted from audio versions of the received signals from the probe. These features are used to train or classify data into clusters.

The functions performed by the servers 708, 710, 712 may be enacted by instructions configured to execute programs or scripts in response to requests from user devices 702, 704. Such a process may include executing one or more applications that may be implemented as one or more scripts or programs written in any appropriate programming language. The server(s) 708, 710, 712 may also include one or more database servers for serving data requests and performing other such operations. The environment 700 can also include any of a variety of data stores and other memory and storage media as discussed above. Where a system includes computerized devices, each such device can include hardware elements that may be electrically coupled via a bus or other such mechanism. Example elements include, as discussed previously, at least one central processing unit (CPU) and one or more storage devices, such as disk drives, optical storage devices, and solid-state storage devices such as random access memory (RAM) or read-only memory (ROM), as well as removable media devices, memory cards, flash cards, etc., as will be readily understood by one of ordinary skill in the art. Such devices can also include or utilize one or more computer-readable storage media for storing instructions executable by at least one processor of the devices. An example device may also include a number of software applications, modules, services, or other elements located in memory, including an operating system and various application programs. It should be appreciated that alternate embodiments may have numerous variations from that described above.

Various types of non-transitory computer-readable storage media can be used for various purposes as discussed and suggested herein. This includes, for example, storing instructions or code that can be executed by at least one processor for causing the system to perform various operations. The media can correspond to any of various types of media, including volatile and non-volatile memory that may be removable in some implementations. The media can store various computer readable instructions, data structures, program modules, and other data or content. Types of media include, for example, RAM, DRAM, ROM, EEPROM, flash memory, solid state memory, and other memory technology. Other types of storage media can be used as well, as may include optical (e.g., Blu-ray or digital versatile disk (DVD)) storage or magnetic storage (e.g., hard drives or magnetic tape), among other such options. Based on the disclosure and teachings provided herein, a person of ordinary skill in the art will appreciate other ways and/or methods to implement the various embodiments.

In some aspects, the present disclosure provides a non-transitory computer-readable medium containing instructions that are executable by one or more processors to perform operations for NUA to detect an allergic reaction. The operations include instructing, via the processor, a US probe communicatively coupled to the processor to transmit a US wave to an area of a subject. The area includes an internal body structure. The operations include receiving, via the processor, a first information describing a reflection of the US wave from the area, generating, via the processor, a second information describing the internal body structure or a characteristic thereof based on processing the first information, generating, via the processor, a third information describing a change in a circulatory system of the subject based on processing the second information, determining, via the processor, a presence of an allergic reaction to a potential allergen in the subject based on the third information, and instructing, via the processor, a user interface communicatively coupled to the processor to output a fourth information indicative of the presence of the allergic reaction. In some embodiments, the third information includes one or more of fifth information describing a change in an immune response associated with the circulatory system, sixth information describing an amount of compensation on the circulatory system, and seventh information describing a change in vascular circulation by the circulatory system. In some embodiments, determining the presence of the allergic reaction includes determining the one or more of the fifth information, the sixth information, and the seventh information is greater than one or more respective thresholds. In some embodiments, generating the second information describing the internal body structure or the characteristic thereof includes determining, for one or more extremities of the subject, an anterograde pulsatility index, an anterograde volume flow, a retrograde pulsatility index, a retrograde volume flow, or a combination thereof and determining a neurovascular index for the one or more extremities based on the anterograde pulsatility index, the anterograde volume flow, the retrograde pulsatility index, the retrograde volume flow, or the combination thereof. In some embodiments, the one or more extremities includes an upper extremity or a lower extremity. In some embodiments, generating the third information includes determining a decrease in a cumulative anterograde volume flow score based on a sum of respective anterograde volume flows of the one or more extremities and generating fifth information describing a change in an immune response associated with the circulatory system based on the decrease in the cumulative anterograde volume flow score. In some embodiments, the third information includes the fifth information. In some embodiments, generating the third information includes determining an increase in an amount of compensation on the circulatory system and generating sixth information describing the amount of compensation based on the increase in the amount of compensation on the circulatory system. In some embodiments, the third information includes the sixth information. In some embodiments, generating the third information includes determining a decrease in an amount of the internal body structure or the characteristic thereof circulating through the circulatory system and generating seventh information describing a change in vascular circulation based on the decrease in the amount of the internal body structure or the characteristic thereof. In some embodiments, the third information includes the seventh information.

The environment in FIG. 7 may be, in one embodiment, a distributed computing environment utilizing several computer systems and components that are interconnected via communication links, using one or more computer networks or direct connections. However, it will be appreciated by those of ordinary skill in the art that such a system could operate equally well in a system having fewer or a greater number of components than are described. Thus, the depictions of various systems and services herein should be taken as being illustrative in nature, and not limiting to the scope of the disclosure.

Various aspects can be implemented as part of at least one service or web service, such as may be part of a service-oriented architecture. Services such as web services can communicate using any appropriate type of messaging, such as by using messages in extensible markup language (XML) format and exchanged using an appropriate protocol such as SOAP (derived from the “Simple Object Access Protocol”). Processes provided or executed by such services can be written in any appropriate language, such as the Web Services Description Language (WSDL). Using a language such as WSDL allows for functionality such as the automated generation of client-side code in various SOAP frameworks.

In embodiments utilizing a server, the server can run any of a variety of server or mid-tier applications, including HTTP servers, FTP servers, CGI servers, data servers, Java servers, and business application servers. The server(s) also may be capable of executing programs or scripts in response requests from user devices, such as by executing one or more Web applications that may be implemented as one or more scripts or programs written in any programming language, such as Java®, C, C# or C++, or any scripting language, such as Perl, Python®, or Tool Command Language (TCL), as well as combinations thereof. The server(s) may also include database servers, including without limitation those commercially available from Oracle®, Microsoft®, Sybase®, and IBM®.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers, or other network devices may be stored locally and/or remotely, as appropriate. Where a system includes computerized devices, each such device can include hardware elements that may be electrically coupled via a bus, the elements including, for example, at least one central processing unit (CPU), at least one input device (e.g., a mouse, keyboard, controller, touch screen, or keypad), and at least one output device (e.g., a display device, printer, or speaker). Such a system may also include one or more storage devices, such as disk drives, optical storage devices, and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.), and working memory as described above. The computer-readable storage media reader can be connected with, or configured to receive, a computer-readable storage medium, representing remote, local, fixed, and/or removable storage devices as well as storage media for temporarily and/or more permanently containing, storing, transmitting, and retrieving computer-readable information. The system and various devices may also include a number of software applications, modules, services, or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets), or both. Further, connection to other computing devices such as network input/output devices may be employed.

The vascular limitations imposed on a single neuron are complex and involve multiple systems that participate in regulating and promoting neurovascular circulation distally and proximally. These systems may have a compromised vascular system distally or locally which may impact the circulation both locally and distally through hypofunction or hyperfunction of the regulatory system both proximally and distally.

In the foregoing description, numerous specific details are set forth, such as specific configurations, dimensions and processes, etc., in order to provide a thorough understanding of the embodiments. In other instances, well-known processes and ultrasound techniques have not been described in particular detail in order to not unnecessarily obscure the embodiments. Reference throughout this specification to “one embodiment,” “an embodiment,” “another embodiment,” “other embodiments,” “some embodiments,” and their variations means that a particular feature, structure, configuration, or characteristic described in connection with the embodiment is included in at least one embodiment. Thus, the appearances of the phrase “for one embodiment,” “for an embodiment,” “for another embodiment,” “in other embodiments,” “in some embodiments,” or their variations in various places throughout this specification are not necessarily referring to the same embodiment. Furthermore, the particular features, structures, configurations, or characteristics may be combined in any suitable manner in one or more embodiments.

In the following description and claims, the terms “coupled” and “connected,” along with their derivatives, may be used. It should be understood that these terms are not intended as synonyms for each other. “Coupled” is used to indicate that two or more elements or components, which may or may not be in direct physical or electrical contact with each other, co-operate or interact with each other. “Connected” is used to indicate the establishment of communication between two or more elements or components that are coupled with each other.

Some portions of the detailed description have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. It should be borne in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as those set forth in the claims below, refer to the action and processes of a computer system, or similar electronic computing system, that manipulates and transforms data represented as physical (electronic) quantities within the computer system's registers and memories into other data similarly represented as physical quantities within the computer system memories or registers or other such information storage, transmission or display devices.

Embodiments described herein can include or relate to an apparatus for performing a computer program (e.g., the operations described herein, etc.). Such a computer program is stored in at least one non-transitory computer readable medium. A machine-readable medium includes any mechanism for storing information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices).

Although one or more embodiments of at least two of the operations or methods set forth above are described in a sequential order, it should be appreciated that any two or more of these operations or methods may be performed in a different order. Moreover, at least two or more of these operations or methods may be performed in parallel rather than sequentially. Embodiments described herein are not described with reference to any particular programming language. It will be appreciated that a variety of programming languages may be used to implement the various embodiments of the disclosed subject matter. In utilizing the various aspects of the embodiments described herein, it should be appreciated that combinations, modifications, or variations of at least one of the embodiments described herein are possible for managing components of a processing system to increase the power and performance of at least one of those components. Thus, various modifications may be made thereto without departing from the broader spirit and scope of at least one of the disclosed concepts set forth in the following claims. The specification and drawings are, accordingly, to be regarded in an illustrative sense rather than a restrictive sense.

In the development of any actual implementation of one or more of the disclosed concepts (e.g., such as a software and/or hardware development project, etc.), numerous decisions must be made to achieve the developers' specific goals (e.g., compliance with system-related constraints and/or business-related constraints). These goals may vary from one implementation to another, and this variation could affect the actual implementation of one or more of the disclosed concepts set forth in the embodiments described herein. Such development efforts might be complex and time-consuming, but may still be a routine undertaking for a person having ordinary skill in the art in the design and/or implementation of one or more of the inventive concepts set forth in the embodiments described herein.

Ultrasound imaging according to the NUA systems, methods, and apparatuses disclosed herein is particularly useful for identifying which systems of a subject may be compromised by a tested allergen, including either proximally or distally positioned systems. Ultrasound imaging may also be used to identify which nerves may have been impacted around an ultrasound probe. As such, ultrasound imaging identifies the systems that impact the neural circulation and the level of any ischemia that is occurring at neurovascular structures proximal to the ultrasound probe. This identification facilitates the quantitative and efficient detection of allergic responses in the subject.

In more detail, monitored allergic reactions generated according to the present embodiments may cause neurovascular inflammation to occur at any point on a specific nerve and result in decreased nerve conduction from that point. Neural conduction generally propels its own endoneurial circulation, with decreased nerve firing resulting in slowed endoneurial circulation and further resulting in decreased nerve firing. In short, the neurovascular inflammation generates a positive feedback loop. Immune intervention of the positive feedback loop may then start, including neural ischemia with fluid leaking from porous capillaries, thus closing the circulation from epineurial capillaries and resulting in perineurial occlusion of transperineural communicating capillaries. The neurovascular index (NVI) of the NUA embodiments disclosed herein facilitates detection of that area creating downstream inflammation.

Neuropathy is a combination of decreased electrical/endoneurial ischemia occurring either upstream or more peripherally, where local resistance increases with decreased outside/inside hydraulic pressure. This decreased hydraulic pressure may often occur in more distal extremities of the subject. The NVI-associated measurements discussed above may be evaluated by a user to evaluate the larger macro-vascular picture or state of the system and/or the smaller vessels or details of the system. When there is an electrical breakdown, such as at the insular cortex, precentral or postcentral cortex, or distal nerve, there will be a separation between proximal and distal nerve readings. The most common separation is a decrease in the more proximal reading and an increase in the more distal reading. The more distal reading generally does not benefit from the strong endoneurial blood flow that is driven by orthograde or anterograde neural firing. The circulation is furnished solely by the local arterial to small vessel push. When that occurs, a separation is generated between the two readings because the hydraulic push closest to the heart will overcome more neural capillary resistance with a deeper circulatory flow. However, the circulation will be more turbulent with back flow into the artery creating a more deficient wave form of the Doppler ultrasound image discussed above for the more proximal area.

In contrast, the more distal area of the subject will have a less deficient wave form because the hydraulic push further from the heart will be lower in intensity. This relationship indicates that, in the absence of a strong endoneurial circulatory system, the epineurial circulation will be more superficial and cleaner while encountering less resistance. However, along with the epineurial circulation, some AV shunting may occur with deeper capillary ischemia. This degree of discrepancy of the NVI reading between the proximal nerve and distal nerve provides the user of the NUA system with an understanding of the extent of endoneurial capillary congestion. The wider the discrepancy, the greater the congestion, and vice versa.

In certain embodiments, examination of the upper extremity (UE) and lower extremity (LE) NVI readings provides a system-level view of circulation within the subject. A user equipped with the NUA embodiments disclosed herein can compare small fiber UE NVI readings to small fiber LE NVI readings, small fiber NVI readings to large fiber NVI readings, and/or refocus examination on individual nerves of choice. Visceral inflammation can also be assessed with the NVI readings, because the inflammation may create ischemia in paravertebral ganglion, which may impact adjacent sympathetic nerves serving the ipsilateral LE. The user can also evaluate nerve root circulation. Certain examples of major systems and problems regarding nerve root circulation that can be identified include autonomic neuropathy with its variable input into the microvascular circulation, small fiber degeneration and its extent, large fiber degeneration and its extent, individual nerves that are affected by the ischemia, and the general inflammation in the entire system and its extent. The NUA method also facilitates examination of the local hydraulic (macrovascular to microvascular) verses systematic/local electrical (endoneurial) causes of neuropathy.

Accordingly, the NVI-based embodiments of the NUA system for allergy detection have the capability of providing an objective, quantifiable estimate as to the degree of inflammation that a person is experiencing in total. A health professional can evaluate the severity of a particular subject's inflammation by addition of the h-factor (epineurial) and e-factor (endoneurial) circulation and identify ischemic bias, such as if the ischemia is primarily on the outside or the inside of a fascicle of a nerve. An addition of both factors provides a total ischemic factor that can be used by the health professional to estimate a degree to which the subject is allergic to a specific, tested allergen. The non-limiting example discussed below illustrates certain use of these factors for allergy detection according to the NUA method disclosed herein.

Indeed, as discussed above, food allergies have traditionally been difficult to diagnose. However, changes recorded in the NVI readings have demonstrated an improved or impaired relationship between the microvascular and macrovascular system following the exposure to certain types of food. As would be understood by one of ordinary skill in the art, the NUA methods and systems disclosed herein may assist with research involving foods or medicines and how they impact the microvascular circulation of certain affected subjects.

Embodiments of the present disclosure can be further understood by reference to the following example.

### EXAMPLES

The present embodiments of neurovascular ultrasound analysis (NUA) facilitate the non-subjective and non-invasive evaluation of a subject for an allergic reaction to allergens. In an embodiment of the present disclosure, a US probe is positioned on or in the area of an extremity of the subject. In the present examples, certain areas of both left upper extremities (LUEs) and left lower extremities (LLEs) are evaluated before exposing the subject to a specific allergen to determine individualized baseline NVI readings for the areas of the subject. The subject was also evaluated after a controlled exposure to the specific allergen to determine post exposure NVI readings. During the baseline and the post exposure tests, a US wave was emitted by the US probe into the area, and the NUA assessment was conducted according to the embodiments of the present disclosure.

As disclosed herein, the methods for NUA facilitate determination of a degree of allergic reaction to a tested allergen. For example, the NUA may indicate the subject has no reaction, a low response, a moderate response, or a severe response to the allergen based on the quantification of the inflammation sensed in the body of the subject. In the present examples discussed below, the severity of the subject's allergic response to the allergen was evaluated with respect to a neurovascular index (NVI) that assess the h-factor (epineurial) and the e-factor (endoneurial) discussed above to identify any ischemic bias. Indeed, the NVI presents and analyzes bodily inflammation information for allergy detection, where increased inflammation readings after allergen exposure indicate an allergic reaction.

In certain embodiments, the NUA assessment evaluates any waveform variability changes in response to the tested allergen that are outside of a normal range. In some of these embodiments, the normal range of NVI is seven to fourteen and the tested areas include the neck, the back, the tibial nerve, and/or the peroneal nerve of the subject. In certain embodiments, the NUA assessment also evaluates any abnormal anterograde volume flow changes, such as increases or decreases that are outside of normal variance. These volume flow changes may include: (1) an increase in anterograde volume flow indicating cardiac changes (e.g., increases) in response to inflammation, such as greater than 1.5 times control, (2) a decrease in anterograde volume flow indicating histamine release in response to the tested allergen, and/or (3) a combination of upper extremity (UI) increase and lower extremity (LE) decrease that indicates pattern changes in relationship to position of a tested area relative to the heart.

As presented below, certain complete or full-spectrum NUA embodiments provide robust allergy detection based on US measurements taken at ten different bodily sites. In certain embodiments, the sites include four UE areas and six LE areas, such as two on a radial side of an arm, two on an ulnar side of the arm, three on a lower medial side of the leg (e.g., tibial), and three on a lower lateral side of the leg (e.g., peroneal). All ten US measurements may be collected before allergen exposure and then again after the allergen exposure.

### Example 1 of Neurovascular Ultrasound Analysis for Allergy Detection

The initial readings and corresponding results for a first tested allergen are set forth in Tables 1-4 below. Table 1 displays the baseline NVI readings for the LUEs and LLEs of the subject. Table 2 displays the post exposure NVI readings for the LUEs and LLEs of the subject. Table 3 displays the individual nerve results for large fiber nerves. Table 4 displays the results of NUA for allergy detection within the subject. The results of Tables 3 and 4 are discussed below.

TABLE 1
Baseline NVI Readings for Left Upper and Left Lower Extremities

| | LUE | H | E | |
|---|---|---|---|---|
| Subclavian | 60.095 | 10.095 | | 125 |
| Brachial | 60.095 | 10.095 | | 125 |
| Prox Radial | 50.873 | 0.873 | 9.221 | 125 |
| Prox Ulnar | 63.525 | 13.525 | 3.431 | 125 |
| Distal Radial | 62.990 | 12.990 | 12.117 | 125 |
| Distal Ulnar | 62.990 | 12.990 | 0.536 | 125 |
| Total: | 360.569 | 60.569 | 25.304 | 750 |
| | 0.481 | 0.101 | 0.063 | |
| | LLE | H | E | |
| Deep Fem | 48.900 | 1.100 | | 125 |
| Common Fem | 48.900 | 1.100 | | 125 |
| Prox Pero | 59.462 | 9.462 | 8.362 | 125 |
| Prox PTA | 6.554 | 43.446 | 42.346 | 125 |
| Distal Pero | 43.049 | 6.951 | 2.511 | 125 |
| Distal PTA | 86.535 | 36.535 | 6.912 | 125 |
| Total: | 293.399 | 98.595 | 60.131 | 750 |
| | 0.391 | 0.164 | 0.150 | |

TABLE 2
Post Exposure NVI Readings for Left
Upper and Left Lower Extremities

| | LUE | H | E | |
|---|---|---|---|---|
| Subclavian | 82.392 | 32.392 | | 125 |
| Brachial | 82.392 | 32.392 | | 125 |
| Prox Radial | 84.984 | 34.984 | 2.591 | 125 |
| Prox Ulnar | 85.099 | 35.099 | 2.707 | 125 |
| Distal Radial | 66.883 | 16.883 | 18.101 | 125 |
| Distal Ulnar | 92.604 | 42.604 | 7.505 | 125 |
| Total: | 494.354 | 194.354 | 30.903 | 750 |
| | 0.659 | 0.324 | 0.077 | |
| | LLE | H | E | |
| Deep Fem | 53.584 | 3.584 | | 125 |
| Common Fem | 53.584 | 3.584 | | 125 |
| Prox Pero | 61.773 | 11.773 | 8.190 | 125 |
| Prox PTA | 42.440 | 7.560 | 3.977 | 125 |
| Distal Pero | 83.372 | 33.372 | 21.599 | 125 |
| Distal PTA | 26.749 | 23.251 | 15.691 | 125 |
| Total: | 321.501 | 83.123 | 49.456 | 750 |
| | 0.429 | 0.139 | 0.124 | |

TABLE 3
Individual Nerve Results for Large Fiber Nerves

| | | | Capillary Congestion/ | |
|---|---|---|---|---|
| | | Severity | AV Shunting | |
| Pre Assessment | | | | |
| Proximal Radial (Proximal Median Nerve) | 0.873 | None | AV Shunting | 11 |
| Proximal Ulnar (Proximal Ulnar Nerve) | 13.525 | Mild | AV Shunting | 10 |
| Distal Radial (Median Nerve into Hand) | 12.990 | Mild | AV Shunting | 10 |
| Distal Ulnar (Ulnar Nerve to hand) | 12.990 | Mild | AV Shunting | 10 |
| Proximal Pero (Peroneal Nerve) | 9.362 | None | Epineurial Swelling | |
| Proximal Tibial (Tibial Nerve) | 43.446 | Severe | Epineurial Swelling | 7 |
| Distal Tib (Lateral and Medial Planter Nerve) | 6.951 | None | Epineurial Swelling | |
| Distal Pero (Top of Foot Peroneal Nerve) | 36.535 | Severe | Epineurial Swelling | 7 |
| | | | Total: | 55 |
| Post Assessment | | | | |
| Proximal Radial (Proximal Median Nerve) | 34.984 | Severe | AV Shunting | 8 |
| Proximal Ulnar (Proximal Ulnar Nerve) | 35.099 | Severe | AV Shunting | 8 |
| Distal Radial (Median Nerve into Hand) | 16.883 | Moderate | AV Shunting | 9 |
| Distal Ulnar (Ulnar Nerve to hand) | 42.604 | Severe | AV Shunting | 8 |
| Proximal Pero (Peroneal Nerve) | 11.773 | Mild | AV Shunting | 10 |
| Proximal Tibial (Tibial Nerve) | 7.560 | None | AV Shunting | 11 |
| Distal Tib (Lateral and Medial Planter Nerve) | 33.372 | Severe | AV Shunting | 8 |
| Distal Pero (Top of Foot Peroneal Nerve) | 23.251 | Moderate | AV Shunting | 9 |
| | | | Total: | 71 |

TABLE 4
Results of NUA for Allergy Detection

| | Baseline | Post Exposure |
|---|---|---|
| LUE | 0.481 | 0.659 |
| LLE | 0.391 | 0.429 |
| UE to LE | 1.229 | 1.538 |
| AV Shunting | −0.229 | −0.538 |
| LUE Total Anterograde | 60.560 | 24.130 |
| LLE Total Anterograde | 64.060 | 77.430 |
| Mean Anterograde UE | 15.140 | 6.033 |
| Mean Anterograde LE | 16.015 | 19.358 |
| Total UE and LE | 124.620 | 101.560 |
| Percent from Control | | 0.815 |
| UE to LE Comparison | | 0.398 |
| LE to LE Comparison | | 1.209 |

In some embodiments, the positive criteria for allergy detection with respect to an individually-generated baseline include determining: (1) a ratio of change between a post exposure total NVI reading and a corresponding baseline total NVI reading increases by greater than 1.5 or decreases below 0.85; (2) UE NVI reading increases by greater than 1.25 after exposure and LE NVI reading decreases by more than 0.8 after exposure; (3) AV shunting changes from UE to LE dominance; (4) total AV shunting changes greater than 0.15; or (5) any combination thereof.

According to Table 4 above, the NUA system determines that the subject has a confirmed allergic response to the tested allergen. For example, the AV shunting changed between the control and the post exposure readings by −0.538÷−0.229=2.349, which is greater than the 0.15 threshold. Additionally, the ratio of change between the post exposure total NVI reading and the corresponding baseline total NVI reading is 101.560÷124.620=0.815, which is below the 0.85 threshold. It should be understood that the allergic response may be confirmed by reaching a single one of the positive criteria. Moreover, as additional tests are performed and more data is compiled, the positive criteria may be adjusted as part of training the above discussed classifier or neural networks of FIGS. 6-7, respectively.

### Example 2 of Neurovascular Ultrasound Analysis for Allergy Detection

Example 2 illustrates an additional implementation of the NUA disclosed herein to analyze the reaction of a subject to a tested potential allergen. That is, Example 2 generally follows the same procedure as Example 1 but provides allergen-specific data quantifying any related inflammation in the body of the subject. In the present embodiment, the tested allergen is bread. In such embodiments, a positive diagnosis indication of bread allergy may represent Celiac disease. In Table 5 displays the baseline NVI readings for the LUEs and LLEs of the subject, prior to ingesting the bread. Table 6 displays the post exposure NVI readings for the LUEs and LLEs of the subject, subsequent to bread ingestion. Table 7 displays certain results of NUA for allergy detection within the subject. The results of Table 7 are discussed below.

TABLE 5
Baseline NVI Readings for Left Upper and Left Lower Extremities

| | LUE | H | E | |
|---|---|---|---|---|
| Subclavian | 71.590 | 21.590 | | 125 |
| Brachial | 71.590 | 21.590 | | 125 |
| Prox Radial | 59.690 | 9.690 | 11.900 | 125 |
| Prox Ulnar | 57.208 | 7.208 | 14.382 | 125 |
| Distal Radial | 100.403 | 50.403 | 40.713 | 125 |
| Distal Ulnar | 69.060 | 19.060 | 11.851 | 125 |
| Total: | 429.542 | 129.542 | 78.847 | 750 |
| | 0.573 | 0.216 | 0.197 | |
| | LLE | H | E | |
| Deep Fem | 79.824 | 29.824 | | 125 |
| Common Fem | 79.824 | 29.824 | | 125 |
| Prox Pero | 69.614 | 19.614 | 10.209 | 125 |
| Prox PTA | 52.906 | 2.906 | 26.918 | 125 |
| Distal Pero | 78.780 | 28.780 | 9.166 | 125 |
| Distal PTA | 117.995 | 67.995 | 65.089 | 125 |
| Total: | 478.942 | 178.942 | 111.383 | 750 |
| | 0.639 | 0.298 | 0.278 | |

TABLE 6
Post Exposure NVI Readings for Left
Upper and Left Lower Extremities

| | LUE | H | E | |
|---|---|---|---|---|
| Subclavian | 70.071 | 20.071 | | 125 |
| Brachial | 70.071 | 20.071 | | 125 |
| Prox Radial | 77.210 | 27.210 | 7.139 | 125 |
| Prox Ulnar | 53.511 | 3.511 | 16.560 | 125 |
| Distal Radial | 71.312 | 21.312 | 5.897 | 125 |
| Distal Ulnar | 78.251 | 28.251 | 24.739 | 125 |
| Total: | 420.426 | 120.426 | 54.335 | 750 |
| | 0.561 | 0.201 | 0.136 | |
| | LLE | H | E | |
| Deep Fem | 71.082 | 21.082 | | 125 |
| Common Fem | 71.082 | 21.082 | | 125 |
| Prox Pero | 81.143 | 31.143 | 10.061 | 125 |
| Prox PTA | 72.746 | 22.746 | 1.665 | 125 |
| Distal Pero | 50.123 | 0.123 | 31.020 | 125 |
| Distal PTA | 80.314 | 30.314 | 7.568 | 125 |
| Total: | 426.489 | 126.489 | 50.314 | 750 |
| | 0.569 | 0.211 | 0.126 | |

TABLE 7
Results of NUA for Allergy Detection

| | Baseline | Post Exposure |
|---|---|---|
| LUE | 0.573 | 0.561 |
| LLE | 0.639 | 0.569 |
| UE to LE | 0.897 | 0.986 |
| AV Shunting | 0.103 | 0.014 |
| LUE Total Anterograde | 49.070 | 45.030 |
| LLE Total Anterograde | 243.700 | 243.610 |
| Mean Anterograde UE | 12.268 | 11.258 |
| Mean Anterograde LE | 60.925 | 60.903 |
| Total UE and LE | 292.770 | 288.640 |
| Percent from Control | | 0.986 |
| UE to LE Comparison | | 0.918 |
| LE to LE Comparison | | 1.000 |

According to Table 7 above, the NUA system may determine that the subject does not have a significant allergic response to the tested allergen in this step of the NUA. As one example, the AV shunting changed between the control and the post exposure readings by 0.014÷0.103=0.136, which is less than the 0.15 threshold. Additionally, as further tests are performed and more data is compiled, the positive criteria may be adjusted as part of training the above discussed classifier or neural networks of FIGS. 6-7, respectively.

Moreover, Table 8 displays standard deviations of left side NVI scores. In the present example, the analyzed nerves of the upper extremity include the prox. radial, prox. ulnar, distal radial, and distal ulnar nerves. The analyzed nerves of the lower extremity include the prox. peroneal, prox. PTA, distal peroneal, and distal PTA nerves. Further, Table 9 displays a 3-point analysis for specific nerves. The standard deviation of the NVI scores is also provided. As shown, a statistically significant change is detected via the NUA after exposure of the subject to the tested allergen. Accordingly, the subject may be reliably diagnosed with an allergy to the tested allergen.

TABLE 8
Standard Deviations of Left Side NVI Scores

| | Baseline | Post Exposure |
|---|---|---|
| UE std | 17.212 | 9.920 |
| LE std | 23.911 | 12.535 |

TABLE 9
3-Point Nerve Specific Analysis

| | Pero | Tibia |
|---|---|---|
| Baseline | | |
| Prox | 69.614 | 52.906 |
| Intermittent | 61.800 | 67.733 |
| Distal | 78.780 | 117.995 |
| std | 6.939 | 27.854 |
| Post Exposure | | |
| Prox | 81.143 | 72.746 |
| Intermittent | 74.958 | 37.013 |
| Distal | 50.123 | 80.314 |
| std | 13.405 | 18.883 |

### Example 3 for Efficacy Statistics of Neurovascular Ultrasound Analysis

Table 10 displays efficacy statistics of certain Intraneural Facilitation treatment (INF™) assessments performed without and with the NVI analysis techniques disclosed herein. As illustrated, a statistical significance of various factors of a Pain Quality Assessment Scale (PQAS) are represented via a P value. The P value is generally lower in the assessments including the NVI analysis, indicating a greater statistical significance for the corresponding observed factors based on the presently disclosed NUA techniques.

TABLE 10
Efficacy Statistics of NUA Excluding Vs. Including NVI Analysis

| PQAS | INF Only - P value | INF + NVI Food testing - P value |
|---|---|---|
| Numbness | 0.013 | 0.00001 |
| Unpleasant | 0.039 | 0.00001 |
| Sharp | 0.019 | 0.00079 |
| Sensitive | Not significant | 0.00003 |
| Radiating | Not significant | 0.01200 |
| Surface | Not significant | 0.00003 |

Prior to the present disclosure, there has been no way to estimate or quantify microvascular circulation. To be more effective, an instrument or apparatus of the present NUA methods and systems may evaluate an entire circulation system and the individual nerves, including both large and small nerves. It may further evaluate certain autonomic dysregulation that is present with neuropathic patients. The NUA methods and systems disclosed herein address these demands, while actively grading and providing a picture of the microvascular system. Indeed, the methods, systems, and apparatuses disclosed herein provide multiple significant benefits over prior allergy evaluation methods, such as non-subjectivity, increased ease, and reduced invasiveness to the subject.

The present disclosure described herein, therefore, is well adapted to carry out the objects and attain the ends and advantages mentioned, as well as others inherent therein. While several specific embodiments have been provided for purposes of disclosure, numerous changes can exist and be made to the details of methods and systems for accomplishing the desired results. These and other similar modifications will readily suggest themselves to those skilled in the art, and are intended to be encompassed within the spirit of the present disclosure disclosed herein and the scope of the appended claims.

What is claimed is:

1. A neurovascular ultrasound (US) analysis system for determining a presence of an allergic reaction in a subject, comprising:
a US probe configured to transmit a US wave to an area of the subject, wherein the area comprises an internal body structure; and
a processor communicatively coupled to the US probe, wherein the processor is configured to execute instructions to:
receive, via the US probe, a first information describing a reflection of the US wave from the area;
generate a second information describing the internal body structure or a characteristic thereof based on processing the first information;
generate a third information describing a change in a circulatory system of the subject based on processing the second information;
determine the presence of the allergic reaction to a potential allergen in the subject based on the third information; and
output, via a user interface communicatively coupled to the processor, a fourth information indicative of the presence of the allergic reaction.

2. The neurovascular US analysis system of claim 1, wherein the third information comprises one or more of:
fifth information describing a change in an immune response associated with the circulatory system;
sixth information describing an amount of compensation on the circulatory system; and
seventh information describing a change in vascular circulation by the circulatory system.

3. The neurovascular US analysis system of claim 1, wherein the processor is configured to execute the instructions to generate the second information by:
determining, for an extremity associated with the area, at least one of an anterograde pulsatility index, an anterograde volume flow, a retrograde pulsatility index, and a retrograde volume flow; and
determining a neurovascular index for the extremity composed of the at least one of the anterograde pulsatility index, the anterograde volume flow, the retrograde pulsatility index, and the retrograde volume flow.

4. The neurovascular US analysis system of claim 3, wherein the processor is configured to execute the instructions to determine an arteriovenous (AV) shunting score for the extremity based on the neurovascular index and a predetermined extremity score.

5. The neurovascular US analysis system of claim 3, wherein the processor is configured to execute the instructions to determine a cumulative anterograde volume flow score based on a sum of respective anterograde volume flows of the extremity and at least one additional extremity of the subject.

6. The neurovascular US analysis system of claim 5, wherein the processor is configured to execute the instructions to generate the third information by:
determining a decrease in the cumulative anterograde volume flow score; and
generating fifth information describing a change in an immune response associated with the circulatory system based on the decrease in the cumulative anterograde volume flow score.

7. The neurovascular US analysis system of claim 1, wherein the processor is configured to execute the instructions to generate the third information by:
determining an increase in an amount of compensation on the circulatory system; and
generating sixth information describing the amount of compensation on the circulatory system based on the increase in the amount of compensation on the circulatory system.

8. The neurovascular US analysis system of claim 1, wherein the processor is configured to execute the instructions to generate the third information by:
determining a decrease in an amount of the internal body structure or the characteristic thereof circulating through the circulatory system; and
generating seventh information describing a change in vascular circulation by the circulatory system based on the decrease in the amount of the internal body structure or the characteristic thereof circulating through the circulatory system.

9. The neurovascular US analysis system of claim 1, wherein the processor is configured to output the fourth information indicative of the presence of the allergic reaction following exposure of the subject to the potential allergen.

10. The neurovascular US analysis system of claim 1, wherein the processor is configured to:
output a pre-exposure fourth information indicative of the presence of the allergic reaction prior to exposure of the subject to the potential allergen to establish baseline measurements; and
output the fourth information indicative of the presence of the allergic reaction following exposure of the subject to the potential allergen and based on the baseline measurements and post-exposure measurements.

11. The neurovascular US analysis system of claim 1, wherein the area of the subject comprises a first area of a plurality of tested areas of the subject, and wherein the plurality of tested areas comprises:
a first area and a second area on a radial side of an arm;
a first area and a second area on an ulnar side of the arm;
a first area, a second area, and a third area on a lower medial side of a leg; and
a first area, a second area, and a third area on a lower lateral side of the leg.

12. The neurovascular US analysis system of claim 1, wherein the area comprises an upper extremity of the subject or a lower extremity of the subject, wherein the upper extremity comprises one or more of a proximal radius, a proximal ulna, a distal radius, and a distal ulna, and wherein the lower extremity comprises one or more of a proximal tibia, a proximal peroneal internal body structure, a distal tibia, and a distal peroneal internal body structure.

13. A non-transitory computer-readable medium comprising instructions that are executable by a processor to perform operations for neurovascular ultrasound (US) analysis to determine a presence of an allergic reaction, the operations comprising:
instructing, via the processor, a US probe communicatively coupled to the processor to transmit a US wave to an area of a subject, wherein the area comprises an internal body structure;
receiving, via the processor, a first information describing a reflection of the US wave from the area;
generating, via the processor, a second information describing the internal body structure or a characteristic thereof based on processing the first information;
generating, via the processor, a third information describing a change in a circulatory system of the subject based on processing the second information;
determining, via the processor, the presence of the allergic reaction to a potential allergen in the subject based on the third information; and
instructing, via the processor, a user interface communicatively coupled to the processor to output a fourth information indicative of the presence of the allergic reaction.

14. The non-transitory computer-readable medium of claim 13, wherein the third information comprises one or more of:
fifth information describing a change in an immune response associated with the circulatory system;
sixth information describing an amount of compensation on the circulatory system; and
seventh information describing a change in vascular circulation by the circulatory system; and
wherein determining the presence of the allergic reaction comprises determining the one or more of the fifth information, the sixth information, and the seventh information is greater than one or more respective thresholds.

15. The non-transitory computer-readable medium of claim 13, wherein generating the second information describing the internal body structure or the characteristic thereof comprises:
determining, for one or more extremities of the subject, an anterograde pulsatility index, an anterograde volume flow, a retrograde pulsatility index, a retrograde volume flow, or a combination thereof, wherein the one or more extremities comprises an upper extremity or a lower extremity; and
determining a neurovascular index for the one or more extremities composed of the anterograde pulsatility index, the anterograde volume flow, the retrograde pulsatility index, the retrograde volume flow, or the combination thereof.

16. The non-transitory computer-readable medium of claim 15, wherein generating the third information comprises:
determining a decrease in a cumulative anterograde volume flow score based on a sum of respective anterograde volume flows of the one or more extremities; and
generating fifth information describing a change in an immune response associated with the circulatory system based on the decrease in the cumulative anterograde volume flow score, wherein the third information comprises the fifth information.

17. The non-transitory computer-readable medium of claim 13, wherein generating the third information comprises:
determining an increase in an amount of compensation on the circulatory system; and
generating sixth information describing the amount of compensation based on the increase in the amount of compensation on the circulatory system, wherein the third information comprises the sixth information.

18. The non-transitory computer-readable medium of claim 13, wherein generating the third information comprises:
determining a decrease in an amount of the internal body structure or the characteristic thereof circulating through the circulatory system; and
generating seventh information describing a change in vascular circulation based on the decrease in the amount of the internal body structure or the characteristic thereof, wherein the third information comprises the seventh information.

19. A computer-implemented method of neurovascular ultrasound (US) analysis (NUA) for determining a presence of an allergic reaction in a subject, comprising:
instructing, via a processor, a US probe communicatively coupled to the processor to transmit a US wave to an area of the subject, wherein the area comprises an internal body structure;
receiving, via the processor, a first information describing a reflection of the US wave from the area;
generating, via the processor, a second information describing the internal body structure or a characteristic thereof based on processing the first information;
generating, via the processor, a third information describing a change in a circulatory system of the subject based on processing the second information, wherein the third information comprises one or more of:
information describing a change in an immune response associated with the circulatory system;
information describing an amount of compensation on the circulatory system; and
information describing a change in vascular circulation;
determining, via the processor, the presence of the allergic reaction to a potential allergen in the subject based on the third information; and
instructing, via the processor, a user interface communicatively coupled to the processor to output a fourth information indicative of the presence of the allergic reaction.

20. The computer-implemented method of claim 19, wherein determining the presence of the allergic reaction comprises determining the one or more of the information describing the change in the immune response, the information describing the amount of compensation, and the information describing the change in vascular circulation is greater than one or more respective thresholds.
